(19) United States
(12) Patent Application Publication
(10) Pub. No.: US 2010/0100148 A1
(43) Pub. Date: Apr. 22, 2010

(54) CAPTURE ASSESSMENT AND OPTIMIZATION OF TIMING FOR CARDIAC RESYNCHRONIZATION THERAPY

(75) Inventors: Xiaoyi Min, Thousand Oaks, CA (US); Jeffery D. Snell, Chatsworth, CA (US)

Correspondence Address: PACESETTER, INC., 15900 VALLEY VIEW COURT, SYLMAR, CA, 91392-9221, US

(73) Assignee: PACESETTER, INC., Sylmar, CA (US)

(21) Appl. No.: 12/255,512

(22) Filed: Oct. 21, 2008

(51) Int. Cl. A61N 1/37

(52) U.S. Cl. 607/27

(57) ABSTRACT

An exemplary method includes performing a ventricular capture assessment, determining a ventricular paced propagation delay (PPD) and/or an interventricular conduction delay (IVCD) using information acquired during the ventricular capture assessment and optimizing at least an interventricular delay (VV) based at least in part on the ventricular paced propagation delay (PPD) and/or the interventricular conduction delay (IVCD). Another exemplary method includes performing an atrial capture assessment, determining an atrial evoked response width (ΔA) and one or more atrio-ventricular intervals (AR) using information acquired during the atrial capture assessment and optimizing an atrio-ventricular (PV or AV) delay based at least in part on the atrial evoked response width (ΔA) and the one or more atrio-ventricular intervals (AR). Other exemplary methods, devices, systems, etc., are also disclosed.

### TECHNICAL FIELD

Subject matter presented herein relates generally to techniques to optimize timing of stimuli for cardiac pacing therapies.

### BACKGROUND

Cardiac resynchronization therapy (CRT) provides an electrical solution to the symptoms and other difficulties brought on by heart failure (HF). CRT can call for delivery of electrical stimuli to the heart in a manner that synchronizes contraction and enhances performance. When CRT delivers stimuli to the right and left ventricles, this is called bi-ventricular pacing. Bi-ventricular pacing aims to improve efficiency of each contraction of the heart and the amount of blood pumped to the body. This helps to lessen the symptoms of heart failure and, in many cases, helps to stop the progression of the disease.

CRT is typically administered via an implantable device such as a pacemaker (e.g., called a CRT-P) or an ICD that has a built-in pacemaker (e.g., called a CRT-D). A CRT-D has the added ability to defibrillate the heart if a patient is at risk for life-threatening arrhythmias. Most traditional ICDs or pacemakers have either one lead placed in the heart's right upper chamber (right atrium, or RA) or the heart's RV, or two leads, placed in the heart's RA and RV. CRT devices typically have three leads: one in the RA, one in the RV, and one in the left ventricle (LV). Such a configuration allows for bi-ventricular pacing.

CRT devices typically include more features than a conventional pacing or ICD device. Some of these features require periodic execution, which can deplete a device's energy and even cause a patient to experience discomfort or sub-optimal therapy. As the number of features increase, a need exists for uncovering and capitalizing on synergies that may exist between various features. Various exemplary technologies disclosed herein aim to meet this need and/or other needs.

### SUMMARY

An exemplary method includes performing a ventricular capture assessment, determining a ventricular paced propagation delay (PPD) and/or an interventricular conduction delay (IVCD) using information acquired during the ventricular capture assessment and optimizing at least an interventricular delay (VV) based at least in part on the ventricular paced propagation delay (PPD) and/or the interventricular conduction delay (IVCD). Another exemplary method includes performing an atrial capture assessment, determining an atrial evoked response width (ΔA) and one or more atrio-ventricular intervals (AR) using information acquired during the atrial capture assessment and optimizing an atrio-ventricular (PV or AV) delay based at least in part on the atrial evoked response width (ΔA) and the one or more atrio-ventricular intervals (AR). Other exemplary methods, devices, systems, etc., are also disclosed.

In general, the various methods, devices, systems, etc., described herein, and equivalents thereof, are optionally suitable for use in a variety of pacing therapies and other cardiac related therapies.

### BRIEF DESCRIPTION OF THE DRAWINGS

Features and advantages of the described implementations can be more readily understood by reference to the following description taken in conjunction with the accompanying drawings.

FIG. 1 is a simplified diagram illustrating an exemplary implantable stimulation device in electrical communication with at least three leads implanted into a patient's heart and at least one other lead for delivering stimulation and/or shock therapy.

FIG. 2 is a functional block diagram of an exemplary implantable stimulation device illustrating basic elements that are configured to provide cardioversion, defibrillation, pacing stimulation or other tissue or nerve stimulation.

FIG. 3 is a block diagram of various exemplary algorithms for capture and timing assessment and an exemplary coordination algorithm to coordinate capture and timing assessments.

FIG. 4 is a block diagram of a capture assessment method and an automatic timing optimization method.

FIG. 5 is a block diagram of a method for setting one or more timings for delivery of cardiac pacing therapy.

FIG. 6 is a block diagram of an exemplary atrial capture method along with an atrial cardiac electrogram and associated information.

FIG. 7 is a block diagram of an exemplary ventricular capture method along with ventricular cardiac electrograms and associated information.

FIG. 8 is a block diagram of an optimization method that relies on various information which may be acquired using capture assessment techniques and/or timing assessment techniques.

FIG. 9 is a block diagram of an exemplary method for acquiring atrial information for capture assessments and/or timing assessments.

FIG. 10 is a block diagram of an exemplary method for acquiring right ventricular information for capture assessments and/or timing assessments.

FIG. 11 is a block diagram of an exemplary method for acquiring left ventricular information for capture assessments and/or timing assessments.

FIG. 12 is a diagram of an exemplary method acquiring information using a multisite ventricular lead.

FIG. 13 is a block diagram of various exemplary methods for determining PV, AV and/or VV values based on a variety of information.

FIG. 14 is an exemplary system that includes an implantable device, a programmer configured to program the implantable device, an ECG unit that may provide data to the programmer, and a data base that may store data generated by any of the various devices.

### DETAILED DESCRIPTION

The following description includes the best mode presently contemplated for practicing the described implementations. This description is not to be taken in a limiting sense, but rather is made merely for the purpose of describing the general principles of the implementations. The scope of the described implementations should be ascertained with reference to the issued claims. In the description that follows, like numerals or reference designators will be used to reference like parts or elements throughout.

### Overview

The AutoCapture™ set of algorithms (St. Jude Medical, Inc., Sylmar, Calif.) is a leading feature in the CRMD device industry for capture assessment and the QuickOpt™ set of algorithms (St. Jude Medical, Inc., Sylmar, Calif.) is a first-to-market feature for CRT optimization. As described herein, various synergies are identified between these two technologies, which may be applied, generally, to many capture and timing assessment techniques. Synergies are described in sensing/pacing configurations and testing so that several tests can be combined, which may result in a hybrid method. Combined algorithms can help reduce clinical risks and patient symptoms, simplify load of routine tests and conserve resources.

Various examples show how information acquired during execution of capture assessment algorithms can be used to optimize timings, especially for delivery of CRT or other multisite pacing therapies (e.g., twin site left ventricular pacing).

An exemplary implantable device is described followed by a summary of capture algorithms and timing algorithms. These algorithms are then described in detail along with techniques to use information acquired during capture assessment to optimize one or more timing parameters. An exemplary system that includes an implantable device programmer is also disclosed.

### Exemplary Stimulation Device

The techniques described below are optionally implemented in connection with any stimulation device that is configured or configurable to stimulate and/or shock tissue. With respect to assessment of cardiac condition, an implantable device may provide for acquiring information and analyzing information to assess cardiac condition even in the instance that the device does not provide for (or is not configured/programmed for) delivery of stimulation therapy.

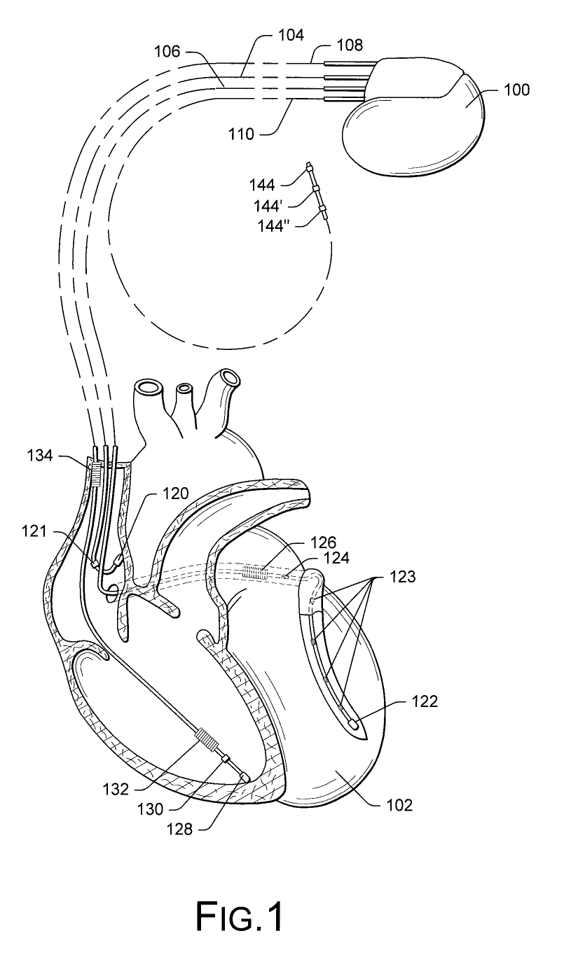

FIG. 1 shows an exemplary stimulation device 100 in electrical communication with a patient's heart 102 by way of three leads 104, 106, 108, suitable for delivering multi-chamber cardiac stimulation and shock therapy. The leads 104, 106, 108 are optionally configurable for delivery of stimulation pulses suitable for stimulation of autonomic nerves, non-myocardial tissue, other nerves, etc. In addition, the device 100 includes a fourth lead 110 having, in this implementation, three electrodes 144, 144′, 144″ suitable for stimulation of any of a variety of tissues (e.g., myocardial, autonomic nerves, non-myocardial tissue, other nerves, etc.). For example, this lead may be positioned in and/or near a patient's heart or near an autonomic nerve within a patient's body and remote from the heart. Such a lead may also include one or more electrodes for epicardial placement (e.g., consider patch, screw, and other attachment mechanisms).

The right atrial lead 104, as the name implies, is positioned in and/or passes through a patient's right atrium. The right atrial lead 104 optionally senses atrial cardiac signals and/or provides for right atrial chamber stimulation therapy. The right atrial lead 104 may be used in conjunction with one or more other leads and/or electrodes to acquire cardiac electrograms and/or to delivery energy to the heart or other tissue. As shown in FIG. 1, the stimulation device 100 is coupled to an implantable right atrial lead 104 having, for example, an atrial tip electrode 120, which typically is implanted in the patient's right atrial appendage. The lead 104, as shown in FIG. 1, also includes an atrial ring electrode 121. Of course, the lead 104 may have other electrodes as well. For example, the right atrial lead optionally includes a distal bifurcation having electrodes suitable for use in stimulation of tissue.

To sense atrial cardiac signals, ventricular cardiac signals and/or to provide chamber pacing therapy, particularly on the left side of a patient's heart, the stimulation device 100 is coupled to a coronary sinus lead 106 designed for placement in the coronary sinus and/or tributary veins of the coronary sinus. Thus, the coronary sinus lead 106 is optionally suitable for positioning at least one distal electrode adjacent to the left ventricle and/or additional electrode(s) adjacent to the left atrium. In a normal heart, tributary veins of the coronary sinus include, but may not be limited to, the great cardiac vein, the left marginal vein, the left posterior ventricular vein, the middle cardiac vein, and the small cardiac vein.

In the example of FIG. 1, the coronary sinus lead 106 includes a series of electrodes 123. In particular, a series of four electrodes are shown positioned in an anterior vein of the heart 102. Other coronary sinus leads may include a different number of electrodes than the lead 106. As described herein, an exemplary method selects one or more electrodes (e.g., from electrodes 123 of the lead 106) and determines characteristics associated with conduction and/or timing in the heart to aid in ventricular pacing therapy and/or assessment of cardiac condition. As described in more detail below, an illustrative method acquires information using various electrode configurations where an electrode configuration typically includes at least one electrode of a coronary sinus lead or other type of left ventricular lead. Such information may be used to determine a suitable electrode configuration for the lead 106 (e.g., selection of one or more electrodes 123 of the lead 106).

An exemplary coronary sinus lead 106 can be designed to receive ventricular cardiac signals (and optionally atrial signals) and to deliver left ventricular pacing therapy using, for example, at least one of the electrodes 123 and/or the tip electrode 122. The lead 106 optionally allows for left atrial pacing therapy, for example, using at least the left atrial ring electrode 124. The lead 106 optionally allows for shocking therapy, for example, using at least the left atrial coil electrode 126. For a complete description of a coronary sinus lead, the reader is directed to U.S. Pat. No. 5,466,254, “Coronary Sinus Lead with Atrial Sensing Capability” (Helland), which is incorporated herein by reference.

The coronary sinus lead 106 further optionally includes electrodes for stimulation of other tissue. Such a lead may include pacing and autonomic nerve stimulation functionality and may further include bifurcations or legs. For example, an exemplary coronary sinus lead includes pacing electrodes capable of delivering pacing pulses to a patient's left ventricle and at least one electrode capable of stimulating an autonomic nerve. An exemplary coronary sinus lead (or left ventricular lead or left atrial lead) may also include at least one electrode capable of stimulating an autonomic nerve, non-myocardial tissue, other nerves, etc., wherein such an electrode may be positioned on the lead or a bifurcation or leg of the lead.

Stimulation device 100 is also shown in electrical communication with the patient's heart 102 by way of an implantable right ventricular lead 108 having, in this exemplary implementation, a right ventricular tip electrode 128, a right ventricular ring electrode 130, a right ventricular (RV) coil electrode 132, and an SVC coil electrode 134. Typically, the right ventricular lead 108 is transvenously inserted into the heart 102 to place the right ventricular tip electrode 128 in the right ventricular apex so that the RV coil electrode 132 will be positioned in the right ventricle and the SVC coil electrode 134 will be positioned in the superior vena cava. Accordingly, the right ventricular lead 108 is capable of sensing or receiving cardiac signals, and delivering stimulation in the form of pacing and shock therapy to the right ventricle. An exemplary right ventricular lead may also include at least one electrode capable of stimulating an autonomic nerve, non-myocardial tissue, other nerves, etc., wherein such an electrode may be positioned on the lead or a bifurcation or leg of the lead. A right ventricular lead may include a series of electrodes, such as the series 123 of the left ventricular lead 106.

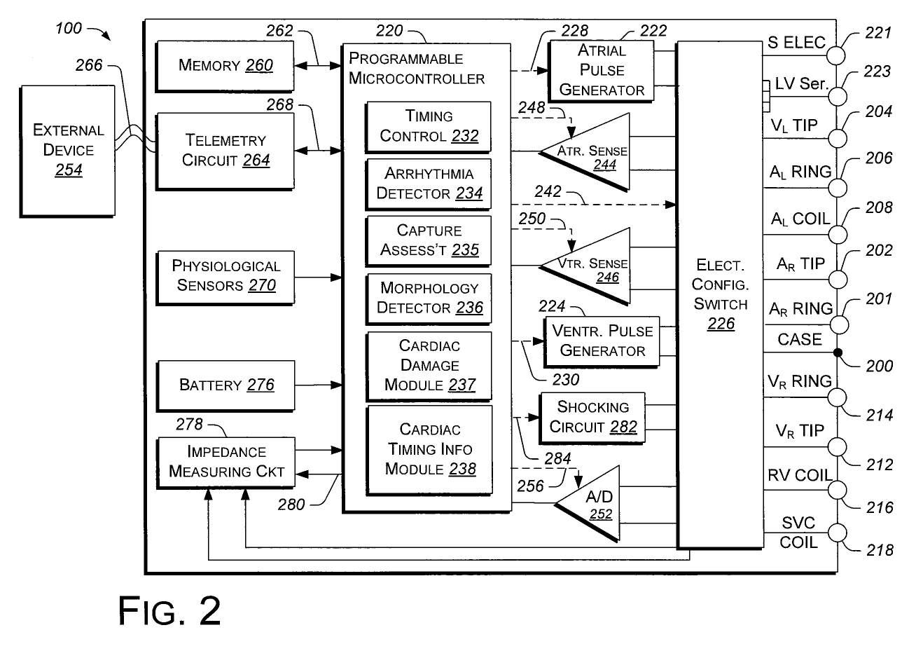

FIG. 2 shows an exemplary, simplified block diagram depicting various components of stimulation device 100. The stimulation device 100 can be capable of treating both fast and slow arrhythmias with stimulation therapy, including cardioversion, defibrillation, and pacing stimulation. The stimulation device can be solely or further capable of delivering stimuli to autonomic nerves, non-myocardial tissue, other nerves, etc. While a particular multi-chamber device is shown, it is to be appreciated and understood that this is done for illustration purposes only. Thus, the techniques and methods described below can be implemented in connection with any suitably configured or configurable stimulation device. Accordingly, one of skill in the art could readily duplicate, eliminate, or disable the appropriate circuitry in any desired combination to provide a device capable of treating the appropriate chamber(s) or regions of a patient's heart with cardioversion, defibrillation, pacing stimulation, autonomic nerve stimulation, non-myocardial tissue stimulation, other nerve stimulation, etc. As already mentioned, for purposes of assessment of cardiac condition, an exemplary implantable device may provide for acquiring information and analyzing such information without delivering stimulation therapy. Hence, an exemplary device may include sensing features without stimulation features or be programmed in a manner where a call for delivery of stimulation therapy does not occur (e.g., prohibited, not enabled, not programmed, etc.).

Housing 200 for stimulation device 100 is often referred to as the “can”, “case” or “case electrode”, and may be programmably selected to act as the return electrode for all “unipolar” modes. Housing 200 may further be used as a return electrode alone or in combination with one or more of the coil electrodes 126, 132 and 134 for shocking purposes. In general, housing 200 may be used as an electrode in any of a variety of electrode configurations. Housing 200 further includes a connector (not shown) having a plurality of terminals 201, 202, 204, 206, 208, 212, 214, 216, 218, 221, 223 (shown schematically and, for convenience, the names of the electrodes to which they are connected are shown next to the terminals).

To achieve right atrial sensing, pacing and/or autonomic stimulation, the connector includes at least a right atrial tip terminal (A_{R} TIP) 202 adapted for connection to the atrial tip electrode 120. A right atrial ring terminal (A_{R} RING) 201 is also shown, which is adapted for connection to the atrial ring electrode 121.

To achieve left chamber sensing, pacing, shocking, and/or autonomic stimulation, the connector includes at least a left ventricular tip terminal (V_{L} TIP) 204, a left atrial ring terminal (A_{L} RING) 206, and a left atrial shocking terminal (A_{L} COIL) 208, which are adapted for connection to the left ventricular tip electrode 122, the left atrial ring electrode 124, and the left atrial coil electrode 126, respectively.

A terminal 223 allows for connection of a series of left ventricular electrodes. For example, the series of four electrodes 123 of the lead 106 may connect to the device 100 via the terminal 223. The terminal 223 and an electrode configuration switch 226 allow for selection of one or more of the series of electrodes and hence electrode configuration. In the example of FIG. 2, the terminal 223 includes four branches to the switch 226 where each branch corresponds to one of the four electrodes 123.

Connection to suitable autonomic nerve stimulation electrodes is also possible via aforementioned terminals and/or other terminals (e.g., via a nerve stimulation terminal S ELEC 221).

To support right chamber sensing, pacing, shocking, and/or autonomic nerve stimulation, the connector further includes a right ventricular tip terminal (V_{R} TIP) 212, a right ventricular ring terminal (V_{R} RING) 214, a right ventricular shocking terminal (RV COIL) 216, and a superior vena cava shocking terminal (SVC COIL) 218, which are adapted for connection to the right ventricular tip electrode 128, right ventricular ring electrode 130, the RV coil electrode 132, and the SVC coil electrode 134, respectively. Connection to suitable autonomic nerve stimulation electrodes is also possible via these and/or other terminals (e.g., via the nerve stimulation terminal S ELEC 221).

At the core of the stimulation device 100 is a programmable microcontroller 220 that controls the various modes of stimulation therapy. As is well known in the art, microcontroller 220 typically includes a microprocessor, or equivalent control circuitry, designed specifically for controlling the delivery of stimulation therapy, and may further include RAM or ROM memory, logic and timing circuitry, state machine circuitry, and I/O circuitry. Typically, microcontroller 220 includes the ability to process or monitor input signals (data or information) as controlled by a program code stored in a designated block of memory. The type of microcontroller is not critical to the described implementations. Rather, any suitable microcontroller 220 may be used that carries out the functions described herein. The use of microprocessor-based control circuits for performing timing and data analysis functions are well known in the art.

Representative types of control circuitry that may be used in connection with the described embodiments can include the microprocessor-based control system of U.S. Pat. No. 4,940,052 (Mann et al.), the state-machine of U.S. Pat. Nos. 4,712,555 (Thornander et al.) and 4,944,298 (Sholder), all of which are incorporated by reference herein. For a more detailed description of the various timing intervals used within the stimulation device and their inter-relationship, see U.S. Pat. No. 4,788,980 (Mann et al.), also incorporated herein by reference.

FIG. 2 also shows an atrial pulse generator 222 and a ventricular pulse generator 224 that generate pacing stimulation pulses for delivery by the right atrial lead 104, the coronary sinus lead 106, and/or the right ventricular lead 108 via the electrode configuration switch 226. One or both of the generators 222 and 224 may optionally provide energy for delivery by the lead 110. It is understood that in order to provide stimulation therapy in each of the four chambers of the heart (or to autonomic nerves or other tissue) the atrial and ventricular pulse generators 222 and 224 may include dedicated, independent pulse generators, multiplexed pulse generators, or shared pulse generators. The pulse generators 222 and 224 are controlled by the microcontroller 220 via appropriate control signals 228 and 230, respectively, to trigger or inhibit the stimulation pulses.

Microcontroller 220 further includes timing control circuitry 232 to control the timing of the stimulation pulses (e.g., pacing rate, atrio-ventricular (AV) delay, atrial interconduction (ΔA) delay, or ventricular interconduction (VV) delay, etc.) as well as to keep track of the timing of refractory periods, blanking intervals, noise detection windows, evoked response windows, alert intervals, marker channel timing, etc., which is well known in the art.

Microcontroller 220 further includes an arrhythmia detector 234, a capture assessment module 235, a morphology detector 236, and optionally an orthostatic compensator and a minute ventilation (MV) response module, the latter two are not shown in FIG. 2. The components can be utilized by the stimulation device 100 for determining desirable times to administer various therapies and for determining appropriate energy levels for delivery of stimuli to the heart. The aforementioned components may be implemented in hardware as part of the microcontroller 220, or as software/firmware instructions programmed into the device and executed on the microcontroller 220 during certain modes of operation.

Microcontroller 220 further includes a cardiac damage module 237 for analyzing information to determine location of one or more cardiac regions or zones, for example, as related to cardiac damage and/or health. The module 237 may use information acquired via one or more of the physiological sensor 270, information acquired via a lead (consider, e.g., leads 104, 106, 108, 110), and/or information acquired via the telemetry circuit 264 (e.g., from an external device). The module 237 may receive information from one or more modules and/or transmit information to one or more modules. The module 237 may act to control various features of the device 100 (e.g., timing of stimulation, timing of sensing, etc.). Module 237 may be implemented in hardware as part of the microcontroller 220, or as software/firmware instructions programmed into the device and executed on the microcontroller 220 during certain modes of operation.

Microcontroller 220 further includes a cardiac timing information module 238 for determining one or more cardiac timing parameters. The module 238 may include logic to determine an intrinsic conduction delay between right ventricular activation and left ventricular activation, an interval between stimulation of one ventricle and sensing of propagated electrical activity to the other ventricle, etc. Module 238 may be implemented in hardware as part of the microcontroller 220, or as software/firmware instructions programmed into the device and executed on the microcontroller 220 during certain modes of operation. The module 238 may operate based in part on analyses performed using the module 237. Further, while the modules are shown as individual modules, other arrangements are possible. The module 238 may operate based in part on information acquired using a capture algorithm or, more generally, a capture assessment method.

As described herein, the module 238 may perform a variety of tasks related to paced propagation delays (PPDs) and/or intervals. A paced propagation delay (PPD) may be considered a “travel” time for a wavefront and may be measured from a delivery time of a stimulus to a feature time as sensed on a wavefront resulting from the stimulus (e.g., a feature of an evoked response). For example, a paced propagation delay may be measured from a delivery time of a right ventricular stimulus to a maximum positive slope (e.g., repolarization) of an evoked response in the right ventricle. Such a delay may be used to help determine one or more parameters for delivery of CRT.

The electronic configuration switch 226 includes a plurality of switches for connecting the desired electrodes to the appropriate I/O circuits, thereby providing complete electrode programmability. Accordingly, switch 226, in response to a control signal 242 from the microcontroller 220, determines the polarity of the stimulation pulses (e.g., unipolar, bipolar, combipolar, etc.) by selectively closing the appropriate combination of switches (not shown) as is known in the art.

Atrial sensing circuits 244 and ventricular sensing circuits 246 may also be selectively coupled to the right atrial lead 104, coronary sinus lead 106, the right ventricular lead 108 and/or the lead 110 through the switch 226 for detecting the presence of cardiac activity in each of the four chambers of the heart. Accordingly, the atrial (ATR. SENSE) and ventricular (VTR. SENSE) sensing circuits 244 and 246 may include dedicated sense amplifiers, multiplexed amplifiers or shared amplifiers. Switch 226 can determine the “sensing polarity” of the cardiac signal by selectively closing the appropriate switches, as is also known in the art. In this way, a clinician may program the sensing polarity independent of the stimulation polarity. An exemplary method may optionally control polarity. For example, the module 237 may include control logic to select an electrode configuration with a particular polarity. The sensing circuits (e.g., 244 and 246) are optionally capable of obtaining information indicative of tissue capture for use by the capture assessment module 235. As described further below, capture information may be used to assess cardiac condition and/or to optimize delivery of a stimulation therapy.

Each sensing circuit 244 and 246 preferably employs one or more low power, precision amplifiers with programmable gain and/or automatic gain control, bandpass filtering, and a threshold detection circuit, as known in the art, to selectively sense the cardiac signal of interest. The automatic gain control enables the device 100 to deal effectively with the difficult problem of sensing the low amplitude signal characteristics of atrial or ventricular fibrillation.

The outputs of the atrial and ventricular sensing circuits 244 and 246 are connected to the microcontroller 220, which, in turn, is able to trigger or inhibit the atrial and ventricular pulse generators 222 and 224, respectively, in a demand fashion in response to the absence or presence of cardiac activity in the appropriate chambers of the heart. Furthermore, as described herein, the microcontroller 220 is also capable of analyzing information output from the sensing circuits 244 and 246 and/or the data acquisition system 252 to determine or detect whether and to what degree tissue capture has occurred and to program a pulse, or pulses, in response to such determinations. The sensing circuits 244 and 246, in turn, receive control signals over signal lines 248 and 250 from the microcontroller 220 for purposes of controlling the gain, threshold, polarization charge removal circuitry (not shown), and the timing of any blocking circuitry (not shown) coupled to the inputs of the sensing circuits 244 and 246, as is known in the art.

Information acquired by any of the sensing circuits (e.g., 244, 246, 252) is optionally used in a control scheme implemented at least in part by the microcontroller 220. For example, the module 237 may use cardiac electrograms acquired via the ventricular sensing circuitry 246 in an analysis that aims to determine location of one or more cardiac regions or zones. In turn, such an analysis may be used by the module 238 to determine timing for delivery of a pacing pulse or pulses.

For arrhythmia detection, the device 100 utilizes the atrial and ventricular sensing circuits 244 and 246 to sense cardiac signals to determine whether a rhythm is physiologic or pathologic. In reference to arrhythmias, as used herein, “sensing” is reserved for the noting of an electrical signal or obtaining data (information), and “detection” is the processing (analysis) of these sensed signals and noting the presence of an arrhythmia. In some instances, detection or detecting includes sensing and in some instances sensing of a particular signal alone is sufficient for detection (e.g., presence/absence, etc.).

The timing intervals between sensed events (e.g., P-waves, R-waves, and depolarization signals associated with fibrillation) are then classified by the arrhythmia detector 234 of the microcontroller 220 by comparing them to a predefined rate zone limit (i.e., bradycardia, normal, low rate VT, high rate VT, and fibrillation rate zones) and various other characteristics (e.g., sudden onset, stability, physiologic sensors, and morphology, etc.) in order to determine the type of remedial therapy that is needed (e.g., bradycardia pacing, anti-tachycardia pacing, cardioversion shocks or defibrillation shocks, collectively referred to as “tiered therapy”).

Cardiac signals are also applied to inputs of an analog-to-digital (A/D) data acquisition system 252. The data acquisition system 252 is configured to acquire cardiac electrogram signals (e.g., intracardiac electrograms or other), convert the raw analog data into a digital signal, and store the digital signals for later processing and/or telemetric transmission to an external device 254. The data acquisition system 252 is coupled to the right atrial lead 104, the coronary sinus lead 106, the right ventricular lead 108 and/or the lead 110 through the switch 226 to sample cardiac signals or other signals (e.g., nerves, etc.) across any pair of desired electrodes.

The microcontroller 220 is further coupled to a memory 260 by a suitable data/address bus 262, wherein the programmable operating parameters used by the microcontroller 220 are stored and modified, as required, in order to customize the operation of the stimulation device 100 to suit the needs of a particular patient. Such operating parameters define, for example, pacing pulse amplitude, pulse duration, electrode polarity, rate, sensitivity, automatic features, arrhythmia detection criteria, and the amplitude, waveshape, number of pulses, and vector of each shocking pulse to be delivered to the patient's heart 102 within each respective tier of therapy. The exemplary device 100 typically includes capabilities to acquire (e.g., sense or otherwise receive) and store a relatively large amount of data (e.g., from the atrial sensing circuitry 244, the ventricular sensing circuitry 246, data acquisition system 252, the one or more physiological sensors 270, the telemetry circuit 264), which data may then be used for subsequent analysis to guide operation of the device 100.

Advantageously, the operating parameters of the implantable device 100 may be non-invasively programmed into the memory 260 through a telemetry circuit 264 in telemetric communication via communication link 266 with the external device 254, such as a programmer, transtelephonic transceiver, or a diagnostic system analyzer. The microcontroller 220 activates the telemetry circuit 264 with a control signal 268. The telemetry circuit 264 advantageously allows intracardiac electrograms and status information relating to the operation of the device 100 (as contained in the microcontroller 220 or memory 260) to be sent to the external device 254 through an established communication link 266.

The stimulation device 100 can further include one or more physiological sensors 270. For example, the device 100 may include a rate-responsive sensor for use in adjusting a pacing stimulation rate according to a sensed activity state (e.g., rest, exercise, etc.) of a patient. The one or more physiological sensors 270 may be capable of acquiring information for use in detecting changes in cardiac output (see, e.g., U.S. Pat. No. 6,314,323, entitled “Heart stimulator determining cardiac output, by measuring the systolic pressure, for controlling the stimulation”, to Ekwall, issued Nov. 6, 2001, which discusses a pressure sensor adapted to sense pressure in a right ventricle and to generate an electrical pressure signal corresponding to the sensed pressure, an integrator supplied with the pressure signal which integrates the pressure signal between a start time and a stop time to produce an integration result that corresponds to cardiac output), detecting changes in the physiological condition of the heart, detecting diurnal changes in activity (e.g., detecting sleep and wake states), etc. Accordingly, the microcontroller 220 may respond by adjusting any of the various pacing parameters (such as rate, ΔA delay, AV delay, VV delay, etc.) at which the atrial and ventricular pulse generators, 222 and 224, generate stimulation pulses. As already mentioned, a device may acquire information and then use the information to assess cardiac condition, regardless of whether the device is configured or programmed to delivery a stimulation therapy.

While shown as being included within the stimulation device 100, it is to be understood that any of the one or more physiological sensors 270 may also be external to the stimulation device 100, yet implanted within or carried by a patient. Examples of physiological sensors that may be implemented in device 100 include known sensors that, for example, sense respiration rate, pH of blood, ventricular gradient, cardiac output, preload, afterload, contractility, hemodynamics, pressure, and so forth. Another sensor that may be used is one that detects activity variance, wherein an activity sensor is monitored diurnally to detect the low variance in the measurement corresponding to the sleep state. For a complete description of the activity variance sensor, the reader is directed to U.S. Pat. No. 5,476,483 (Bornzin et al.), issued Dec. 19, 1995, which patent is hereby incorporated by reference.

More specifically, the one or more physiological sensors 270 optionally include sensors for detecting movement and minute ventilation in the patient. The one or more physiological sensors 270 may include a position sensor and/or a minute ventilation (MV) sensor to sense minute ventilation, which is defined as the total volume of air that moves in and out of a patient's lungs in a minute. Signals generated by the position sensor and MV sensor are passed to the microcontroller 220 for analysis in determining whether to adjust the pacing rate, etc. The microcontroller 220 monitors the signals for indications of the patient's position and activity status, such as whether the patient is climbing upstairs or descending downstairs or whether the patient is sitting up after lying down.

The one or more physiological sensors 270 may include a pressure sensor. Pressure sensors for sensing left atrial pressure are discussed in U.S. Patent Application US2003/0055345 A1, to Eigler et al., which is incorporated by reference herein. The discussion pertains to a pressure transducer permanently implantable within the left atrium of the patient's heart and operable to generate electrical signals indicative of fluid pressures within the patient's left atrium. According to Eigler et al., the pressure transducer is connected to a flexible electrical lead, which is connected in turn to electrical circuitry, which includes digital circuitry for processing electrical signals. Noted positions of the transducer include within the left atrium, within a pulmonary vein, within the left atrial appendage and in the septal wall.

The exemplary device 100 optionally includes a connector capable of connecting a lead that includes a pressure sensor. For example, the connector 221 optionally connects to a pressure sensor capable of receiving information pertaining to chamber pressures or other pressures.

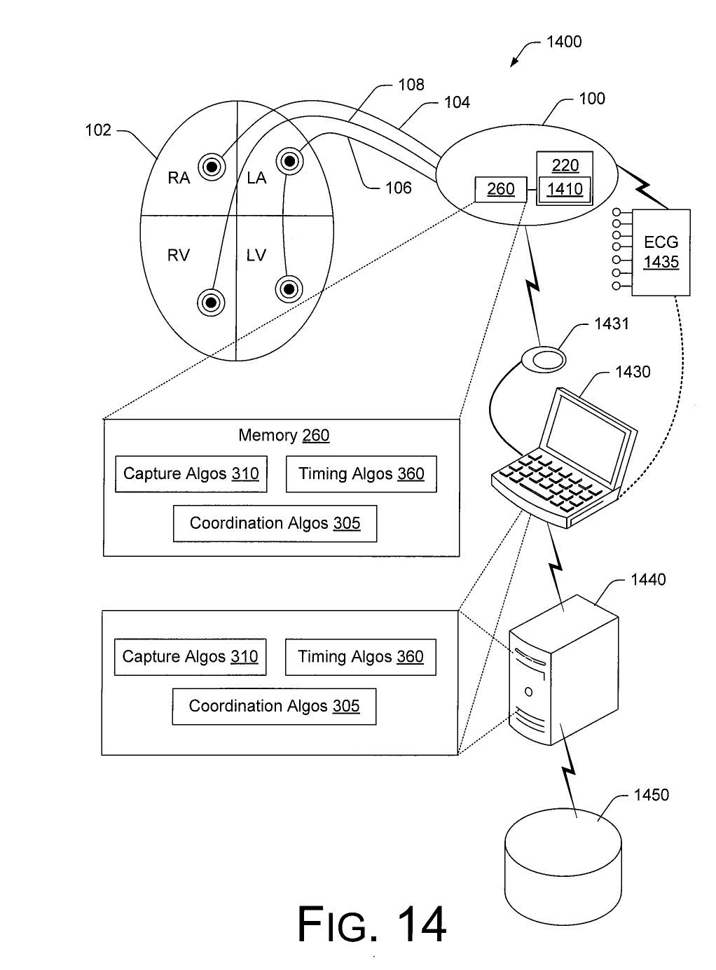

The one or more physiological sensors 270 optionally include an oxygen sensor. The companies Nellcor (Pleasanton, Calif.) and Masimo Corporation (Irvine, Calif.) market pulse oximeters that may be used externally (e.g., finger, toe, etc.). Where desired, information from such external sensors may be communicated wirelessly to the implantable device using appropriate circuitry such as that found in a programmer for an implantable device (see, e.g., the programmer 1430 of FIG. 14).

The exemplary device 100 optionally includes a connector capable of connecting a lead that includes a sensor for sensing oxygen information. For example, the connector 221 optionally connects to a sensor for sensing information related blood oxygen concentration. Such information is optionally processed or analyzed by any of the various modules.

The stimulation device 100 optionally includes circuitry capable of sensing heart sounds and/or vibration associated with events that produce heart sounds. Such circuitry may include an accelerometer as conventionally used for patient position and/or activity determinations. Accelerometers typically include two or three sensors aligned along orthogonal axes. For example, a commercially available micro-electromechanical system (MEMS) marketed as the ADXL330 by Analog Devices, Inc. (Norwood, Mass.), is a small, thin, low power, complete three axis accelerometer with signal conditioned voltage outputs, all on a single monolithic IC. The ADXL330 product measures acceleration with a minimum full-scale range of ±3 g. It can measure the static acceleration of gravity in tilt-sensing applications, as well as dynamic acceleration resulting from motion, shock, or vibration. Bandwidths can be selected to suit the application, with a range of 0.5 Hz to 1,600 Hz for X and Y axes, and a range of 0.5 Hz to 550 Hz for the Z axis. Various heart sounds include frequency components lying in these ranges. The ADXL330 is available in a small, low-profile, 4 mm×4 mm×1.45 mm, 16-lead, plastic lead frame chip scale package (LFCSP_LQ).

While an accelerometer may be included in the case of an implantable pulse generator device, alternatively, an accelerometer communicates with such a device via a lead or through electrical signals conducted by body tissue and/or fluid. In the latter instance, the accelerometer may be positioned to advantageously sense vibrations associated with cardiac events. For example, an epicardial accelerometer may have improved signal to noise for cardiac events compared to an accelerometer housed in a case of an implanted pulse generator device.

As described herein, ischemia, injury and/or infarct may be detectable by various changes in physiology and hence by any of a variety of physiologic sensors, which can include use of aforementioned leads 104, 106, 108, 110 as electrical activity sensors. Ischemia, injury and/or infarct may be detectable based on temperature changes, decreased local myocardial pressure, decreased myocardial pH, decreased myocardial pO₂, increased myocardial pCO₂, increased myocardial lactate, increased ratio of lactate to pyruvate in the myocardium, increased ratio of the reduced form of nicotine amide adenine dinucleotide (NADH) to nicotine amide adenine dinucleotide (NAD⁺) in the myocardium, increased ratio of the reduced form of nicotinamine-adenine dinucleotide phosphate (NADPH) to nicotinamine-adenine dinucleotide phosphate (NADPH) in the myocardium, increased ST segment, decreased ST segment, ventricular tachycardia, T wave changes, QRS changes, decreased patient activity, increased respiratory rate, decreased transthoracic impedance, decreased cardiac output, increased pulmonary artery diastolic pressure, increased myocardial creatinine kinase, increased troponin, and changed myocardial wall motion. Sensed information pertaining to ischemia, injury and/or infarct as well as exemplary mechanisms for sensing such information is discussed in more detail below.

The stimulation device additionally includes a battery 276 that provides operating power to all of the circuits shown in FIG. 2. For the stimulation device 100, which employs shocking therapy, the battery 276 is capable of operating at low current drains for long periods of time (e.g., preferably less than 10 μA), and is capable of providing high-current pulses (for capacitor charging) when the patient requires a shock pulse (e.g., preferably, in excess of 2 A, at voltages above 2 V, for periods of 10 seconds or more). The battery 276 also desirably has a predictable discharge characteristic so that elective replacement time can be detected.

The stimulation device 100 can further include magnet detection circuitry (not shown), coupled to the microcontroller 220, to detect when a magnet is placed over the stimulation device 100. A magnet may be used by a clinician to perform various test functions of the stimulation device 100 and/or to signal the microcontroller 220 that the external programmer 254 is in place to receive or transmit data to the microcontroller 220 through the telemetry circuits 264.

The stimulation device 100 further includes an impedance measuring circuit 278 that is enabled by the microcontroller 220 via a control signal 280. The known uses for an impedance measuring circuit 278 include, but are not limited to, lead impedance surveillance during the acute and chronic phases for proper lead positioning or dislodgement; detecting operable electrodes and automatically switching to an operable pair if dislodgement occurs; measuring respiration or minute ventilation; measuring thoracic impedance for determining shock thresholds, edema, heart failure or other indicators; detecting when the device has been implanted; measuring stroke volume; and detecting the opening of heart valves; etc. The impedance measuring circuit 278 is advantageously coupled to the switch 226 so that any desired electrode may be used.

In the case where the stimulation device 100 is intended to operate as an implantable cardioverter/defibrillator (ICD) device, it detects the occurrence of an arrhythmia, and automatically applies an appropriate therapy to the heart aimed at terminating the detected arrhythmia. To this end, the microcontroller 220 further controls a shocking circuit 282 by way of a control signal 284. The shocking circuit 282 generates shocking pulses of low (e.g., up to approximately 0.5 J), moderate (e.g., approximately 0.5 J to approximately 10 J), or high energy (e.g., approximately 11 J to approximately 40 J), as controlled by the microcontroller 220. Such shocking pulses are applied to the patient's heart 102 through at least two shocking electrodes, and as shown in this embodiment, selected from the left atrial coil electrode 126, the RV coil electrode 132, and/or the SVC coil electrode 134. As noted above, the housing 200 may act as an active electrode in combination with the RV electrode 132, or as part of a split electrical vector using the SVC coil electrode 134 or the left atrial coil electrode 126 (i.e., using the RV electrode as a common electrode). Other exemplary devices may include one or more other coil electrodes or suitable shock electrodes (e.g., a LV coil, etc.).

Cardioversion level shocks are generally considered to be of low to moderate energy level (where possible, so as to minimize pain felt by the patient), and/or synchronized with an R-wave and/or pertaining to the treatment of tachycardia. Defibrillation shocks are generally of moderate to high energy level (i.e., corresponding to thresholds in the range of approximately 5 J to approximately 40 J), delivered asynchronously (since R-waves may be too disorganized), and pertaining exclusively to the treatment of fibrillation. Accordingly, the microcontroller 220 is capable of controlling the synchronous or asynchronous delivery of the shocking pulses.

The device 100 may be configured to delivery cardiac resynchronization therapy. In general, cardiac resynchronization therapy delivers stimulation to improve overall cardiac function. This may have the additional beneficial effect of reducing the susceptibility to life-threatening tachyarrhythmias. CRT and related therapies are discussed in, for example, U.S. Pat. No. 6,643,546 (Mathis et al.), entitled “Multi-Electrode Apparatus and Method for Treatment of Congestive Heart Failure”; U.S. Pat. No. 6,628,988 (Kramer et al.) entitled “Apparatus and Method for Reversal of Myocardial Remodeling with Electrical Stimulation”; and U.S. Pat. No. 6,512,952 (Stahmann et al.), entitled “Method and Apparatus for Maintaining Synchronized Pacing,” which are incorporated by reference herein. An exemplary implantable CRT device optionally includes electrodes for epicardial placement. For example, the lead 110 may include one or more electrodes for epicardial placement.

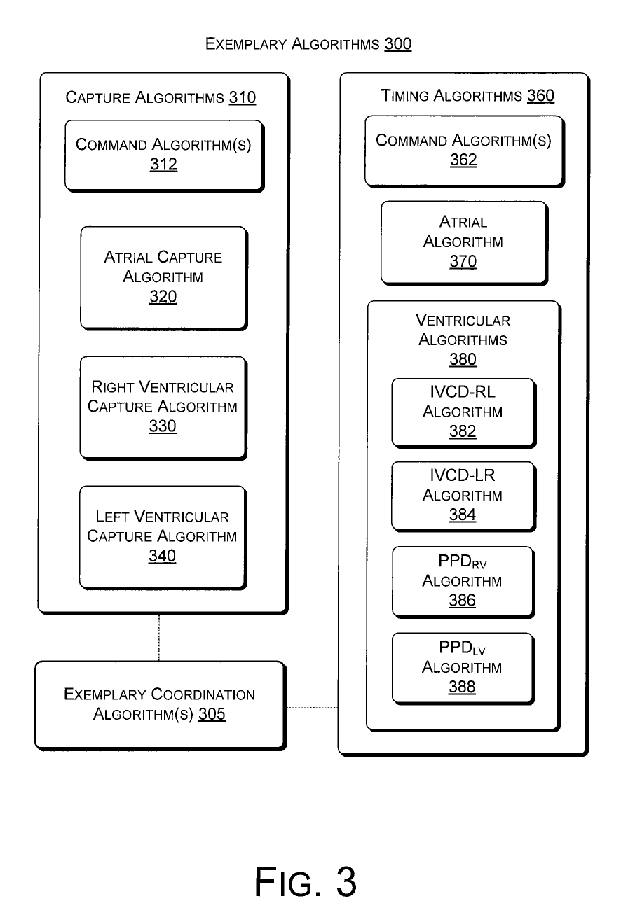

FIG. 3 shows various algorithms 300 for use in adjusting one or more parameters for bi-ventricular pacing and/or cardiac resynchronization therapy. The algorithms 300 are classified as capture algorithms 310, timing algorithms 360 and one or more exemplary coordination algorithms 305. The capture algorithms 310 generally aim to ensure that energy delivered to the heart is sufficient to generate an evoked response. The timing algorithms 360 generally aim to effectively time delivery of energy to the heart. The one or more coordination algorithms 305 allow for coordination of the capture algorithms 310 and the timing algorithms 360. Such coordination may be with respect to triggers (e.g., event-based triggers), scheduling, sharing of information, etc. The algorithms 300 may be considered as ensuring proper timing and reliable capture of energy delivered to the heart.

The one or more coordination algorithms 305 may be considered as ensuring that information is used effectively, for example, to reduce test time, to reduce number of tests, to reduce energy usage, etc., as associated with various algorithms. The one or more coordination algorithms 305 may include instructions to override triggering and/or scheduling mechanisms of capture algorithms 312 and/or to override triggering and/or scheduling mechanisms of timing algorithms 362.

As described herein, an exemplary device includes one or more coordination algorithms (e.g., control logic) that allow information acquired using one or more capture algorithms to be used by one or more timing algorithms. Such a device may include the coordination algorithm(s) 305, the capture algorithms 310 and the timing algorithms 360. The one or more coordination algorithms may also allow for coordinating execution of capture algorithms and timing algorithms, especially where information acquired by a capture algorithm can assist in assessing one or more timing parameters of the timing algorithms.

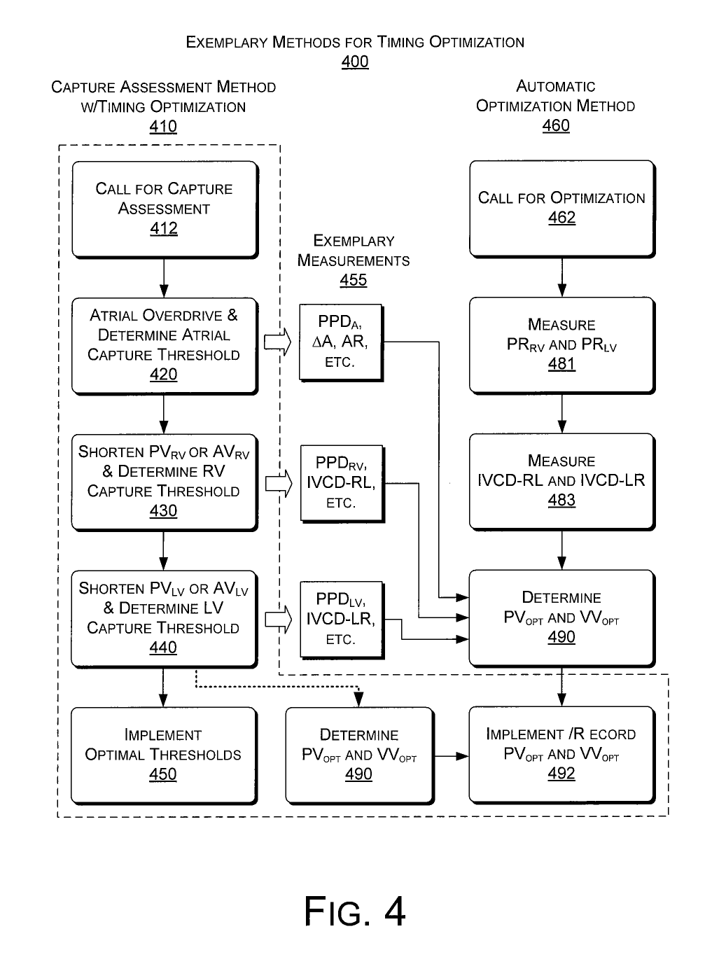

As shown in the example of FIG. 3, the capture algorithms 310 include a command algorithm(s) 312, an atrial capture algorithm 320, a right ventricular capture algorithm 330 and a left ventricular capture algorithm 340. The command algorithm(s) 312 may determine what events can trigger a capture algorithm and may set a schedule for assessing any of a variety of capture thresholds and appropriate energy levels (e.g., atrial, right ventricular, left ventricular, etc.). Various aspects of these algorithms are described in more detail below (see, e.g., FIGS. 4, 6 and 7).

An atrial capture algorithm 320 may include, for example, features of a commercially available atrial capture algorithm marketed as the ACap™ algorithm (St. Jude Medical Inc., Sylmar, Calif.). This algorithm is a confirm feature, which periodically verifies the amount of energy needed for the upper chambers of the heart (atria) to respond to stimulation pulses emitted by a pacing device. Based on the results of this periodic check, a device can automatically self-adjust the energy output required to cause capture.

The right ventricular capture algorithm 330 and the left ventricular capture algorithm 340 may include, for example, features of a commercially available set of algorithms for ventricular capture referred to as the Beat-by-Beat Ventricular AutoCapture™ algorithms (St. Jude Medical, Inc., Sylmar, Calif.). These algorithms include the following features: (a) automatic capture verification, which monitors every beat for the presence of an evoked response (e.g., the signal resulting from electrical activation of the myocardium by a delivered stimulus); (b) automatic stimulation threshold search, which measures myocardial activation thresholds on a regular basis to determine output energy level requirement to readily achieve capture; (c) loss of capture recovery, which triggers an automatic delivery of energy as a backup to ensure capture in the absence of an evoked response; and (d) automatic output regulation, which sets the output energy just above the measured threshold energy level to help ensure that a low and reliable energy level for capture is used (e.g., to help optimize battery longevity). Such algorithms may be used with any of a variety of lead configurations. For example, capabilities for unipolar leads are provided in addition to capabilities for standard bipolar leads.

As shown in the example of FIG. 3, the timing algorithms 360 include a command algorithm(s) 362, an atrial algorithm 370 and ventricular algorithms 380, which include an interventricular conduction delay (IVCD) algorithms 382 and 384 and paced propagation delay (PPD) algorithms 386 and 388, for right and left ventricles, respectively. The command algorithm(s) 362 may determine what events can trigger a timing algorithm and may set a schedule for assessing any of a variety of timings (e.g., atrial, right ventricular, left ventricular, etc.). Various aspects of these algorithms are described in more detail below (see, e.g., FIGS. 4, 5 and 8).

The algorithms of FIG. 3 typically operate based on information in cardiac electrograms (e.g., IEGMs). An IEGM can include information to determine capture, paced propagation delay, etc. Paced propagation delay may be defined generally as the difference between the delivery time of an electrical stimulus and a time associated with a feature of an evoked response (ER), for example, a minimum in amplitude for an ER, maximum slope of an ER, etc.

Various studies have related cardiac electrograms to damage. For example, subendocardial ischemia can prolong local recovery time. Since repolarization normally proceeds in an epicardial-to-endocardial direction, delayed recovery in the subendocardial region due to ischemia does not reverse the direction of repolarization but merely lengthens it. This generally results in a prolonged QT interval or increased amplitude of the T wave or both as recorded by the electrodes overlying, or otherwise sensing activity at, the subendocardial ischemic region. As described herein, a cardiac electrogram may be analyzed for evidence of myocardial damage. A long paced propagation delay, a high capture threshold, a long interventricular conduction delay, etc., can serve as indicators of myocardial damage.

The timing algorithm 360 may include, for example, features of a commercially available set of algorithms for timing of delivered stimuli to the heart are referred to as the QuickOpt™ algorithms (St. Jude Medical, Inc., Sylmar, Calif.). Such algorithms allow for clinician optimization and automatic, device triggered optimization of parameters including AV timing and VV timing. For example, the QuickOpt™ algorithms allow a clinician to program timing(s) (e.g., in about 90 s) so an implantable device can deliver optimal therapy to a patient. The QuickOpt™ algorithms also allow for timing cycle optimization to produce results clinically-proven to be comparable to echocardiography in a significantly less costly and time consuming manner. For example, a typical echocardiography procedure takes between about 30 minutes and about 120 minutes and requires interpretation by a technician; whereas, a QuickOpt™ algorithms optimization allows for frequent optimizations for patients as their needs change (e.g., event triggered, schedule-based, etc.).

Another feature referred to as the Ventricular Intrinsic Preference algorithm (VIP® algorithm, St. Jude Medical, Inc., Sylmar, Calif.) can operate in conjunction with the QuickOpt™ algorithms to reduce unnecessary ventricular pacing. The VIP® algorithm can allow a patient's heart rhythm to prevail when appropriate. The VIP® technology actively monitors the heart on a beat-by-beat basis to provide pacing only when needed, which has been shown in some studies to be better for a patient's overall heart health.

FIG. 4 shows exemplary methods for timing optimization 400. The methods 400 include a capture assessment method 410 and an automatic optimization method for timings 460. The capture assessment method 410 may take about 3 minutes to execute and the automatic optimization method 460 may take about 3 minutes (e.g., depending on implantable device processing capabilities). Hence, a patient may be paced sub-optimally for about 6 minutes if both of the methods 410 and 460 operate separately without coordination. As described herein, various opportunities exist for a hybrid or a combined approach to capture assessment and timing optimization. Such approaches can diminish sub-optimal pacing time. For example, a combined approach may take about 4 minutes versus 6 minutes for a separate approach.

As shown, the capture assessment method 410 can optionally acquire data for one or more measurements 455 for timing optimization. For example, data acquired by the method 410 during a capture assessment may be used alternatively or additionally by the method 460. Such a scheme can alleviate one or more measurements of the method 460. In another arrangement, the capture assessment method 410 may rely on acquired data for timing optimization to thereby yield a combined or hybrid method for capture assessment and timing optimization (e.g., as outlined by dashed line).

FIG. 4 shows the capture method 410 with an optional determination block 490 and implementation block 492, which are explained with respect to the timing optimization method 460. The capture method 410 commences in a call block 412 that calls for capture assessment, which in the example of FIG. 4 assesses capture for atrial, right ventricular and left ventricular activation. As shown, an atrial assessment block 420 includes overdriving the atrial intrinsic rate and then determining the atrial capture threshold. This assessment can acquire data sufficient for measurement of an atrial PPD, atrial waveform width (e.g., ΔA), atrial to right ventricle conduction time (e.g., AR_{RV}) and atrial to left ventricular conduction time (AR_{LV}). A right ventricular assessment block 430 follows that includes shortening the PV_{RV} or AV_{RV} time and then determining the right ventricular capture threshold. This assessment can acquire data sufficient for measurement of a right ventricular PPD and IVCD-RL. A left ventricular assessment block 430 then follows that includes shortening the PV_{LV} or AV_{LV} time and then determining the left ventricular capture threshold. This assessment can acquire data sufficient for measurement of a left ventricular PPD and IVCD-LR. At some point or points in time during the execution of the method 410, the optimal thresholds are implemented, as indicated by an implementation block 450. Optionally, the method 410 may continue to the determination block 490 to determine one or more timings such as PV_{Opt} (or AV_{Opt}) and VV_{Opt} (e.g., for bi-ventricular pacing).

In the example of FIG. 4, the method 460 delivers CRT with periodic optimization according to a specific set of algorithms. A call block 462 calls for optimization according to a schedule, a signal, a cardiac condition, etc. (see, e.g., the block 362 of FIG. 3). As part of the optimization procedure, a measurement block 481 measures a PR_{RV} interval (or AR_{RV}) and a PR_{LV} interval (or AR_{LV}) while another measurement block 483 measures an interventricular conduction delay from the right ventricle to the left ventricle (IVCD-RL) and an interventricular conduction delay from the left ventricle to the right ventricle (IVCD-LR). In general, to measure IVCD-RL or IVCD-LR a stimulus is delivered to one ventricle and a conducted wavefront is sensed in the other ventricle. Such an IVCD may be referred to as a paced IVCD. Alternatively, a sensed IVCD may be used where an intrinsic event is sensed in one ventricle and a conducted wavefront associated with the sensed intrinsic event is sensed in the other ventricle. In either instance, the IVCD provides information about directional conduction between the ventricles. While FIG. 4 shows PV or PR in various blocks, AV or AR may be substituted where appropriate.

As indicated by the aforementioned measurements 455, where available, the method 460 may forego one or more of the measurements of the blocks 481 and 483. For example, where the atrial capture assessment block 420 provides for AR_{RV}, AR_{LV} or both AR_{RV} and AR_{LV}, then the method 460 may forego one or both measurements of the block 481. Similarly, where the RV capture assessment block 430 provides for IVCD-RL, the block 483 need not perform the IVCD-RL measurement and where the LV capture assessment block 440 provides for IVCD-LR, the block 483 need not perform the IVCD-LR measurement. As explained herein, PPD_{A}, PPD_{RV} and PPD_{LV} may be used to optimize one or more timings (or other purpose).

According to the methods 410 and 460, a determination block 490 relies on the measured PR_{RV}, PR_{LV}, IVCD-RL and IVCD-LR values to determine an optimum PV delay (PV_{Opt}) and an optimum VV delay (VV_{Opt}). Such measurements may originate from capture assessment blocks of the method 410, timing optimization blocks of the method 460 or a combination of blocks from the method 410 and the method 460. An implementation block 492 implements the optimized delays PV_{Opt} and VV_{Opt}. In general, such timings are recorded by an implantable device (e.g., for comparison or analysis).

With respect to the method 460, as may be appreciated, if the measurement block 481 cannot measure PR_{RV} or PR_{LV}, the determination block 490 may not be able to determine PV_{Opt} and/or VV_{Opt}. Similarly, if the measurement block 483 cannot accurately measure IVCD-RL or IVCD-LR, the determination block 490 may not function or function improperly. As described herein, various cardiac conditions can confound measurements such as those presented in measurement blocks 481 and 483. In some instances, one or more alternative algorithms or techniques are available to estimate these measures or to optimize PV_{Opt} and/or VV_{Opt}. Some of these techniques have been explained with respect to the measurements 455. Consequently, a patient may be able to benefit from a “restricted” or alternative method to optimize one or more CRT parameters.

As described herein and shown in FIG. 4, various capture algorithms of the capture assessment method 410 may be capable of acquiring information for use by the automatic optimization method 460. Or, alternatively, the capture assessment method 410 may provide for timing optimization per the determination block 490 and implementation block 492.

With respect to parameters used in optimization or delivery of a cardiac therapy, such parameters may include:

PP, AA Interval between successive atrial events
PV Delay between an atrial event and a paced ventricular event
PVₒₚₜᵢₘₐₗ Optimal PV delay
PV_{RV} PV delay for right ventricle
PV_{LV} PV delay for left ventricle
AV Delay for a paced atrial event and a paced ventricular event
AVₒₚₜᵢₘₐₗ Optimal AV delay
AV_{RV} AV delay for right ventricle
AV_{LV} AV delay for left ventricle
Δ Estimated interventricular delay (e.g., AV_{LV}−AV_{RV})
Δ_{programmed} Programmed interventricular delay (e.g., a programmed VV delay)
Δₒₚₜᵢₘₐₗ Optimal interventricular delay
IVCD-RL Delay between an RV event and a consequent sensed LV event
IVCD-LR Delay between an LV event and a consequent sensed RV event
Δ_{IVCD} Difference in interventricular conduction delays (IVCD-LR−IVCD-RL)
ΔP, ΔA Width of an atrial event

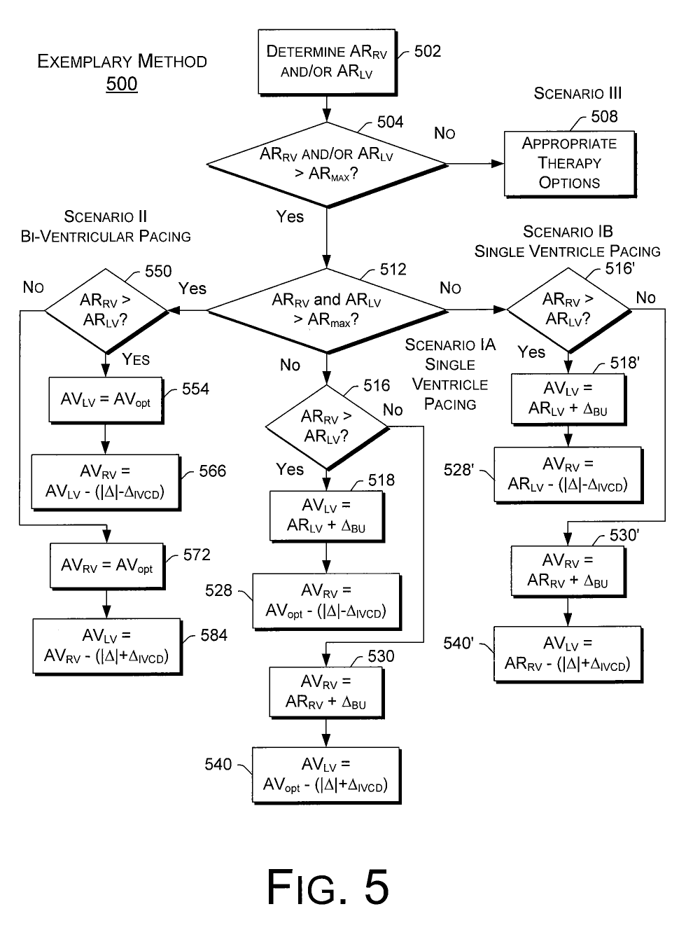

FIG. 5 shows a block diagram of an exemplary method 500, which may be viewed as a more elaborate form of the method 460 of FIG. 4. Specifically, the method 500 illustrates a variety of algorithms germane to optimization of CRT. While the method 500 pertains to atrial pacing, such a method may omit atrial pacing (e.g., rely on an intrinsic atrial activity, etc.) and/or include atrial pacing and intrinsic atrial activity, etc. (e.g., PR, AR, AV, and/or PV). The exemplary method 500 includes Scenarios IA, IB, II and III.

According to the method 500, a determination block 502 determines AR_{RV} and/or AR_{LV}. In a decision block 404 a decision is made as to whether AR_{RV} and/or AR_{LV} have exceeded a predetermined ARₘₐₓ value. If neither value exceeds ARₘₐₓ, then Scenario III follows, which may disable ventricular pacing or take other appropriate therapy options per block 508. Other appropriate therapy optionally includes therapy that achieves a desirable VV delay by any of a variety of techniques. As mention with respect to FIG. 4, such options may be considered restricted or alternative options to standard optimization algorithms and may include acquisition of information using one or more capture algorithms (see, e.g., the capture algorithms 310 of FIG. 3).

In decision block 504, if one or both values exceed ARₘₐₓ, then the method 500 continues in another decision block 512. The decision block 512 decides whether AR_{RV} and AR_{LV} have exceeded ARₘₐₓ. If both values do not exceed ARₘₐₓ, then single ventricular pacing occurs, for example, per Scenario IA or Scenario IB. If both values exceed ARₘₐₓ, then bi-ventricular pacing occurs, for example, Scenario II.

Scenario IA commences with a decision block 516 that decides if AR_{RV} is greater than AR_{LV}. If AR_{RV} exceeds AR_{LV}, then single ventricular pacing occurs in the right ventricle (e.g., right ventricle master). If AR_{RV} does not exceed AR_{LV}, then single ventricular pacing occurs in the left ventricle (e.g., left ventricle master).

For right ventricular pacing per Scenario IA, the method 500 continues in a back-up pacing block 518 where AV_{LV} is set to AR_{LV} plus some back-up time (e.g., Δ_{BU}). The block 518, while optional, acts to ensure that pacing will occur in the left ventricle if no activity occurs within some given interval. The method 500 then continues in a set block 528 where the parameter Δ_{IVCD} is used as a correction factor to set the AV_{RV} delay to AVₒₚₜᵢₘₐₗ−(|Δ|−Δ_{IVCD}).

For left ventricular pacing per the Scenario IA, the method 500 continues in a back-up pacing block 530 where AV_{RV} is set to AR_{RV} plus some back-up time (e.g., Δ_{BU}). The block 530, while optional, acts to ensure that pacing will occur in the left ventricle if no activity occurs within some given interval. The method 500 then continues in a set block 540 where the parameter Δ_{IVCD} is used as a correction factor to set the AV_{LV} delay to AVₒₚₜᵢₘₐₗ−(|Δ|+Δ_{IVCD}). The parameter Δ_{IVCD} is calculated as the difference between IVCD-LR and IVCD-RL (e.g., IVCD-LR−IVCD-RL).

Scenario IB commences with a decision block 516′ that decides if AR_{RV} is greater than AR_{LV}. If AR_{RV} exceeds AR_{LV}, then single ventricular pacing occurs in the right ventricle (e.g., right ventricle master). If AR_{RV} does not exceed AR_{LV}, then single ventricular pacing occurs in the left ventricle (e.g., left ventricle master).

For right ventricular pacing per Scenario IB, the method 500 continues in a back-up pacing block 518′ where AV_{LV} is set to AR_{LV} plus some back-up time (e.g., Δ_{BU}). The block 518′, while optional, acts to ensure that pacing will occur in the left ventricle if no activity occurs within some given interval. The method 500 then continues in a set block 528′ where the parameter Δ_{IVCD} is used as a correction factor to set the AV_{RV} delay to AR_{LV}−(|Δ|−Δ_{IVCD}). Hence, in this example, a pre-determined AVₒₚₜᵢₘₐₗ is not necessary.

For left ventricular pacing per the Scenario IB, the method 500 continues in a back-up pacing block 530′ where AV_{RV} is set to AR_{RV} plus some back-up time (e.g., Δ_{BU}). The block 530′, while optional, acts to ensure that pacing will occur in the left ventricle if no activity occurs within some given interval. The method 500 then continues in a set block 540′ where the parameter Δ_{IVCD} is used as a correction factor to set the AV_{LV} delay to AR_{RV}−(|Δ|+Δ_{IVCD}). Again, in this example, a pre-determined AVₒₚₜᵢₘₐₗ is not necessary.

Referring again to the decision block 512, if this block decides that bi-ventricular pacing is appropriate, for example, Scenario II, then the method 500 continues in a decision block 550, which that decides if AR_{RV} is greater than AR_{LV}. If AR_{RV} exceeds AR_{LV}, then bi-ventricular pacing occurs wherein the right ventricle is the master (e.g., paced prior to the left ventricle or sometimes referred to as left ventricle slave). If AR_{RV} does not exceed AR_{LV}, then bi-ventricular pacing occurs wherein the left ventricle is the master (e.g., paced prior to the right ventricle or sometimes referred to as right ventricle slave).

For right ventricular master pacing, the method 500 continues in a set block 554 which sets AV_{LV} to AVₒₚₜᵢₘₐₗ. The method 500 then uses Δ_{IVCD} as a correction factor in a set block 566, which sets AV_{RV} delay to AV_{LV}−(|Δ|−Δ_{IVCD}).

For left ventricular master pacing, the method 500 continues in a set block 572 which sets AV_{RV} to AVₒₚₜᵢₘₐₗ. The method 500 then uses Δ_{IVCD} as a correction factor in a set block 484, which sets AV_{LV} delay to AV_{RV}−(|Δ|+Δ_{IVCD}).

A comparison between Δ and Δ_{programmed} or Δₒₚₜᵢₘₐₗ can indicate a difference between a current cardiac therapy or state and a potentially better cardiac therapy or state. For example, consider the following equation:

α=Δₒₚₜᵢₘₐₗ/Δ

where α is an optimization parameter. Various echocardiogram studies indicate that the parameter α is typically about 0.5. The use of such an optimization parameter is optional. The parameter α may be used as follows:

AV_{RV}=AVₒₚₜᵢₘₐₗ−α|Δ| or PV_{RV}=PVₒₚₜᵢₘₐₗ−α|Δ|

AV_{LV}=AVₒₚₜᵢₘₐₗ−α(|Δ|+Δ_{IVCD}) or

PV_{LV}=PVₒₚₜᵢₘₐₗ−α(|Δ|+Δ_{IVCD})

If a parameter such as the aforementioned α parameter is available, then such a parameter is optionally used to further adjust and/or set one or more delays, as appropriate.

Various exemplary methods, devices, systems, etc., may consider instances where normal atrio-ventricular conduction exists for one ventricle. For example, if an atrio-ventricular conduction time for the right ventricle does not exceed one or more limits representative of normal conduction, then the atrio-ventricular time for the right ventricle may serve as a basis for determining an appropriate time for delivery of stimulation to the left ventricle (or vice versa). The following equation may be used in such a situation:

AV_{LV}=AR_{RV}−|Δ| or PV_{LV}=PR_{RV}−|Δ|

This equation is similar to the equation used in blocks 528′ and 540′ of Scenario IB of FIG. 5. With respect to backup pulses, a backup pulse (e.g., for purposes of safety, etc.) may be set according to the following equation:

AV_{RV}=AR_{RV}+|γ| or PV_{RV}=PR_{RV}+|γ|

Of course, administration of a backup pulse may occur upon one or more conditions, for example, failure to detect activity in the particular ventricle within a given period of time. In the foregoing equation, the parameter γ is a short time delay, for example, of approximately 5 ms to approximately 10 ms. This equation is similar to the equation used in blocks 518′ and 530′ of Scenario IB of FIG. 5.

In many instances, cardiac condition will affect AR_{RV} and AR_{LV}, and IVCD (e.g., IVCD-RL and/or IVCD-LR), which, in turn, may affect an existing optimal VV delay setting. As explained with respect to the method 460 of FIG. 4, various exemplary methods, devices, systems, etc., include calling for or triggering an algorithm to update an existing optimal VV delay according to a predetermined time or event period or activity sensors for exercise, resting, etc.

As described herein, various techniques can be used to optimize CRT, including capture assessment techniques. Optimization may, at times, rely on use of external measurement or sensing equipment (e.g., echocardiogram, etc.). Further, use of internal measurement or sensing equipment for sensing pressure or other indicators of hemodynamic performance may be optional. Adjustment and learning may rely on IEGM information and/or cardiac other rhythm information.

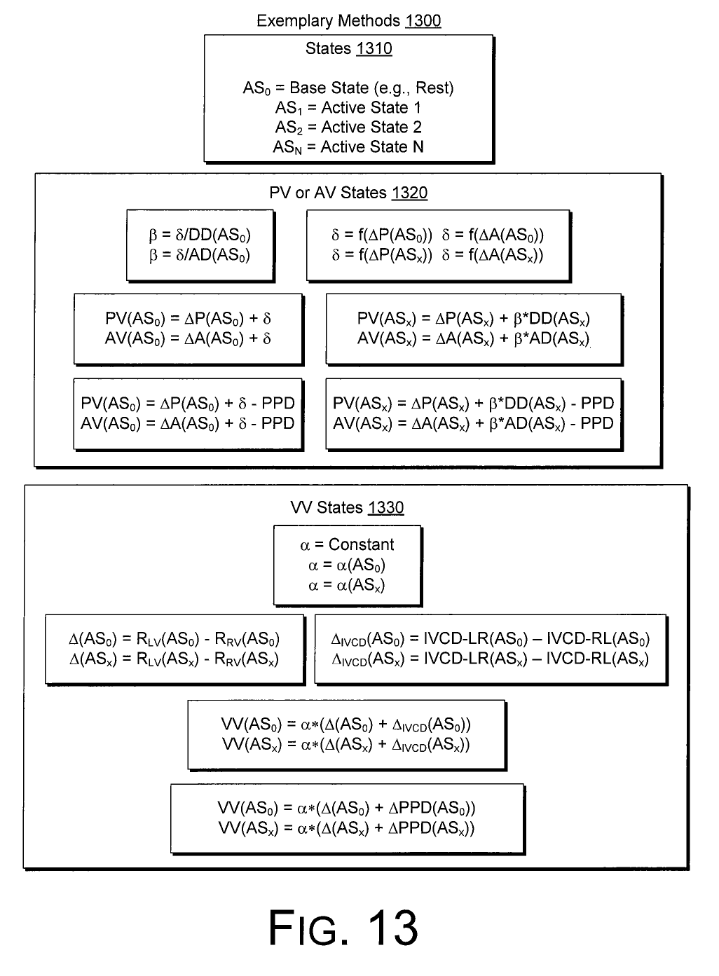

While not indicated in FIG. 5, optimization may rely on atrial information such as width of an atrial wave, time between end of an atrial wave and beginning of a ventricular wave, etc. Such measures may vary with respect to cardiac condition, activity state of a patient, etc. FIG. 13 shows a summary of various activity related states where atrial information may be used to optimize one or more timings.

Atrial information may include beginning of a P wave (P₀) and end of a P wave (P_{End}), where the duration of the P wave or P wave width (ΔP) is P_{End}−P₀. Atrial information may include an interval (DD) between the end of the P wave (P_{End}) and the beginning of an R wave or a QRS complex, for example, as detected by a conventional algorithm or other suitable technique. While “P wave” is mentioned, similar techniques may be used to acquire “A wave” information (e.g., A₀, A_{End}, ΔA, AD based on A_{End} and beginning of an R wave or a QRS complex).

An R wave detection technique may rely on a slope or other feature of an R wave and a time other than the “beginning” of an R wave may be used. A DD interval may rely on a detection technique used for R wave detection. As a DD interval relies on detection of an R wave or a QRS complex, an atrial to ventricular conduction pathway should exist for at least one ventricle because for patients with atrial to ventricular conduction block of both ventricles (e.g., RBBB and LBBB), a meaningful DD interval may not exist. For such patients, measurement of A wave width or P wave width may occur and such values may be used along with activity information for any of a variety of purposes (e.g., cardiac condition, pacing optimization, etc.).

As already mentioned, a PR interval typically relies on detecting P₀, the beginning of a P wave. In contrast, the interval DD relies on detecting P_{End}, the end of a P wave or approximate end of a P wave. Hence, the PR interval is always less than the DD interval for a particular ventricle, noting that one ventricle may have a DD interval that exceeds a PR interval of the other ventricle.

A comparison between rest state electrograms and exercise state electrograms may indicate trends in that the P or A wave duration (ΔP, ΔA) and the DD or AD interval increase with increasing activity. Under normal circumstances, while the AA interval is controlled (e.g., set to a constant or adjusted with respect to activity or other variable), the ratio of A wave duration and AD interval to AA interval or RR interval may be expected to increase.

While aforementioned atrial variables may change with respect to activity, other variables such as PR and Δ may also change with respect to activity. For example, the PR interval may increase where the increase depends on the points used to define the PR interval. However, with respect to Δ, the change may be somewhat uncertain, especially if little data exists for a patient or the patient's condition has changed.

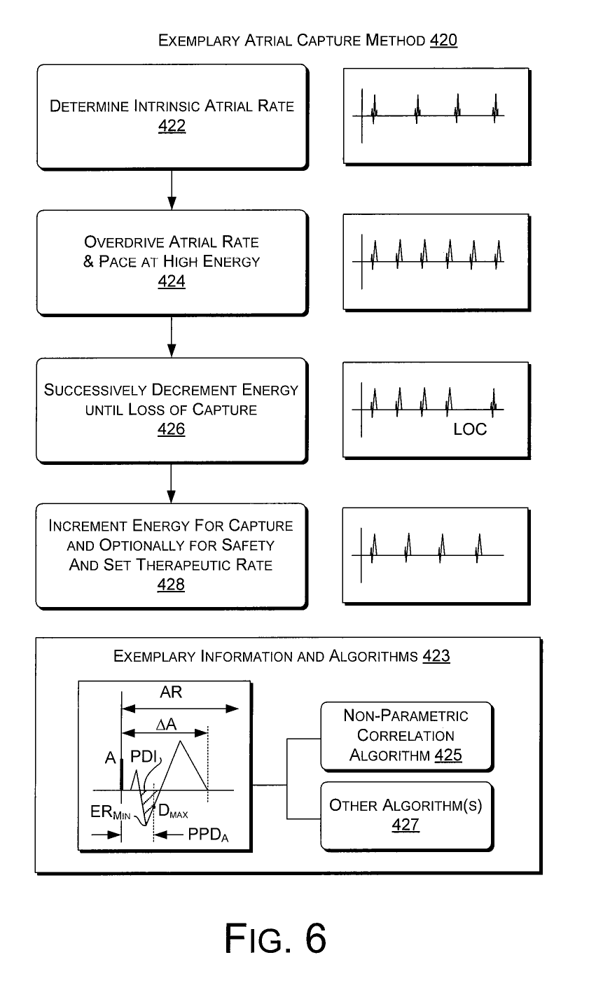

Details of various timing algorithms have been shown in FIG. 5 and described. With respect to details of various capture algorithms, FIG. 6 shows an exemplary atrial capture method 420. The method 420 is shown along with cardiac electrogram information of intrinsic P waves and evoked responses or A waves. The method 420 may include various features of the aforementioned ACap™ algorithm, which can measure a patient's daily atrial capture threshold and adjust atrial output energy accordingly.

The method 420 commences in a determination block 422 that determines an intrinsic atrial rate (e.g., P to P). An overdrive block 424 overdrives the intrinsic atrial rate by pacing faster than the intrinsic atrial rate and at an energy level sufficient to capture the atrium. Hence, an electrogram shows A waves occurring at a rate that exceeds the intrinsic rate.

A decrement block 426 decrements the energy until loss of capture (LOC) occurs. Loss of capture may be indicated by failure to detect an evoked response (A wave) and/or by presence of intrinsic activity (P wave). Once LOC occurs, an increment block 428 increments the energy to a level sufficient to regain capture and it may also optionally adjust the energy for purposes of safety (to reliably ensure capture). Further, the atrial rate may be adjusted to a therapeutic rate. For a patient that is not atrial pacing dependent, the rate may be set to a rate above an acceptable intrinsic rate for the patient.

While the method 420 has been described generally, some specifics of the ACap™ algorithm are provided below. The ACap™ algorithm includes three main steps when performing an automatic threshold test.

In a first step, the algorithm causes an implantable device to pace the atrium faster than the intrinsic atrial rate (see, e.g., block 424). Overdrive atrial pacing minimizes fusion beats and ensures atrial pacing for the threshold test. Specifically, the algorithm causes the implantable device to assess the atrial rate by passively watching for 16 beats (see, e.g., block 422); then, the device paces the atrium faster than the intrinsic rate.

In a second step, the algorithm assesses atrial threshold. Specifically, the algorithm determines where the patient loses capture (see, e.g., block 426). Atrial pacing begins at an operating voltage (high voltage) and decrements by 0.25 V until the device detects three consecutive non-captured beats at the same voltage. Backup pulses are provided during threshold searches to ensure patient safety. Starting at the voltage where capture was lost, the algorithm causes the device to increases atrial output by 0.125 V until two consecutive beats are captured. The algorithms can also call for storage of a weekly threshold trend and follow-up EGMs.

In a third step, the algorithm determines the atrial output. The algorithm sets the atrial output at a fixed voltage above the threshold ensuring an appropriate safety margin.

The ACap™ algorithms also allow for set-up and programmability of various features. For example, prior to activating the ACap™ feature a user can run an automatic ACap™ set-up test. Further, the ACap™ algorithm can be programmed to search every 8 or 24 hours. Yet further, the ACap™ algorithm's thresholds can also be checked in-clinic via an implantable device programmer.

As mentioned, a capture algorithm may provide information for use by one or more timing algorithms. To explain in more detail, exemplary information and algorithms 423 are shown in FIG. 6. An atrial waveform as acquired in the form of a cardiac electrogram can provide information such as PDI, D_{Max}, ER_{Min}, paced propagation delay (PPD_{A}), ΔA, AR_{RV}, AR_{LV}, etc. With respect to AR intervals, for patients without any significant conduction block, the AV delay may be extended (e.g., set well beyond 100 ms) to allow for conduction of an atrial stimulus to one or both ventricles (e.g., depending on condition of conduction path). Such information may be used to determine one or more timing parameters.

When making determinations as to whether atrial capture occurred or not, an algorithm may rely on an integral or integrals (e.g., PDI), a slope or slopes (D_{Max}), etc. A particular non-parametric correlation algorithm 425 relies on a nonparametric measure of association based on the number of concordances and discordances in paired observations (e.g., Kendall tau). In such a technique, concordance occurs when paired observations vary together, and discordance occurs when paired observations vary differently. The Kendall tau rank correlation coefficient (or simply the Kendall tau coefficient, Kendall's t or tau test(s)) is a non-parametric statistic used to measure the degree of correspondence between two rankings and assessing the significance of this correspondence. As indicated in FIG. 6, one or more other algorithms may be used 427 to distinguish capture from non-capture.

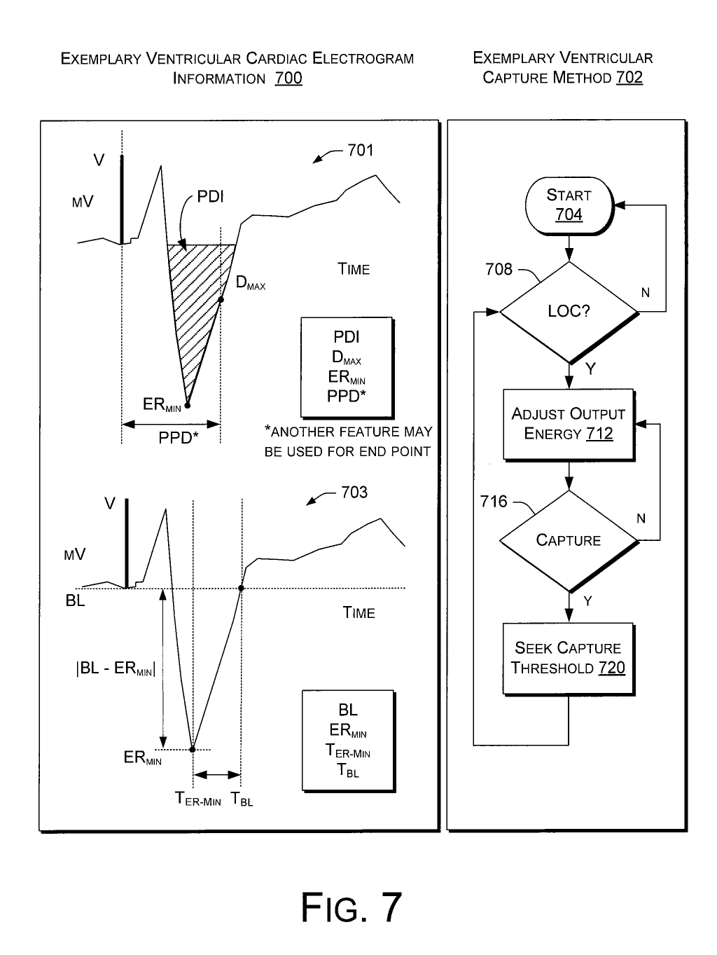

FIG. 7 shows some exemplary ventricular cardiac electrogram information 700 and an exemplary ventricular capture method 702. The information 700 is shown with respect to a first cardiac electrogram 701 and a second cardiac electrogram 703 for ease of explanation as all of the information may be acquired from a single cardiac electrogram. The cardiac electrogram 701 shows measures PDI, D_{Max}, ER_{Min} and PPD while the cardiac electrogram 703 shows a baseline (BL) and a time from a minimum (ER_{Min}) to a return to BL. These examples of FIG. 7 demonstrate how information acquired during a ventricular capture assessment may be used to determine any of a variety of measures. Again, such information or measures may be used by one or more timing algorithms, as explained with respect to FIG. 3.

In more detail, the cardiac electrogram 701 shows timing of a pulse to a ventricle (V) and a corresponding evoked response (ER). The shape of the evoked response depends on a variety of factors, including sensing configuration. For example, sensing polarity may cause the evoked response to be inverted from the shape shown in FIG. 7. In general, for the example of FIG. 7, the evoked response has a minimum ERₘᵢₙ and a maximum slope Dₘₐₓ. These features may be used in conjunction with the timing of V to determine a paced propagation delay as associated with the ventricular site used to deliver the stimulus V. In the example of FIG. 7, paced propagation delay for the ventricle (PPD) is given as Dₘₐₓ−V. Similarly, such information may be acquired for the other ventricle; noting that another ventricular site, etc., could be used depending on the nature of the pacing therapy and lead and electrode configuration.

As described herein, in some instances paced propagation delay (PPD) may be used as a surrogate for IVCD. For example, the difference between the left and right ventricular pacing latencies (ΔPPD) may be used as an estimate for the difference between the IVCD-LR and IVCD-RL (Δ_{IVCD}). A paced propagation delay (PPD) assessment may be used when IVCD-LR and/or IVCD-RL cannot be accurately measured (e.g., due to conduction problems). As a capture algorithm may acquire information sufficient to determine paced propagation delay, measurement of IVCD-RL and/or IVCD-LR may not be required. Or, where IVCD-RL and/or IVCD-LR cannot be measured, then paced propagation delay based on a capture assessment algorithm may be used to determine a surrogate or surrogates for use in determining one or more timings (see, e.g., FIG. 5).

The method 702 illustrates a basic threshold search that relies on capture detection, which may be part of a beat-to-beat or other capture detection algorithm. Again, where capture is not detected, i.e., loss of capture, corrective action is typically required, for example, a change in pulse amplitude, a change in pulse duration, etc.

The method 702 commences in a start block 704, where an implantable device may be programmed to perform capture detection and a threshold search. In some instances, a threshold search is performed on a periodic basis, whether loss of capture has been detected or not. Such a threshold search may help ensure adequate capture as well as battery life. The method 702 continues in a decision block 708 that decides if loss of capture occurred based on sensed cardiac activity. If the decision block 708 decides that loss of capture did not occur, then the method continues at the start block 704. However, if loss of capture occurred, then the method 702 continues at an adjustment block 712 that adjusts energy delivery. For example, the adjustment block 712 may increase amplitude of a stimulation pulse and/or increase duration of a stimulation pulse.

Another decision block 716 follows that decides if a pulse delivered using the adjusted energy caused capture. If the decision block decides that capture did not occur, then the method 702 returns to the adjustment block 712, or it may take other action. However, if capture did occur, then the method 702 continues at a search block 720 that seeks a capture threshold, for example, based on acquired data for prior attempts. After the threshold search 720, the method 702 may return to the decision block 708 or it may take other action as appropriate. In general, such algorithms place patient safety ahead of battery current drain; however, when the chronic threshold is low, such an algorithm may also minimize battery current drain, effectively increasing device longevity.

In addition to beat-to-beat capture verification, the AutoCapture™ algorithm runs a capture threshold assessment test once every eight hours (or other interval, as programmed). To perform this test, the paced and sensed AV delays are temporarily shortened to about 50 ms and to about 25 ms, respectively. The AutoCapture™ algorithm generally uses a bottom-up approach (also referred to as an “up threshold”) and a back-up pulse for safety when an output pulse does not result in capture. With respect to use of a back-up pulse, an output pulse of about 4.5 volts is typically sufficient to achieve capture where lead integrity is not an issue. Use of a back-up pulse may also adequately benefit certain patients that are quite sensitive to loss of capture. For example, patients having a high grade AV block may be sensitive to protracted asystole. Even if loss of capture is recognized immediately and adjustment is completed in less than about 1 second, a patient may still have been asystolic for over 2 seconds utilizing a standard capture threshold test without a back-up. A back-up pulse typically prevents occurrence of such a long asystolic period. However, most conventional automatic capture threshold/detection algorithms do not attempt to detect the evoked response directly related to capture of the back-up. Thus, the assumption that a back-up pulse resulted in capture is generally not tested. Various exemplary methods described herein optionally include evoked response detection of the back-up pulse to help determine if a back-up pulse caused an evoked response thus confirming the presence of capture associated with this stimulus. Such ER or capture detection may be implemented for a unipolar back-up pulse, a bipolar back-up pulse or other type of back-up pulse.

In general, the term “sensing” is often utilized with respect to an implantable cardiac stimulation therapy device (e.g., a pacemaker) recognizing native atrial and/or ventricular depolarizations. While technically, detection of an evoked response (ER) relies on or includes “sensing”, an implantable device often uses a separate circuit for ER detection. Throughout, the term “ER detection” or “capture detection” may be used in place of sensing when specifically concerned with, for example, a capture algorithm and recognition of capture.

With respect to ER detection, various exemplary methods may use a unipolar primary pulse with bipolar ER detection, a unipolar primary pulse with unipolar ER detection, a bipolar primary pulse with bipolar ER detection, a bipolar primary pulse with unipolar ER detection and/or no primary pulse ER detection. Various exemplary methods may use a unipolar back-up pulse with bipolar ER detection, a unipolar back-up pulse with unipolar ER detection, a bipolar back-up pulse with bipolar ER detection, a bipolar back-up pulse with unipolar ER detection and/or back-up pulse ER detection.

Regarding AutoCapture™ algorithms, the first generation algorithm was implemented using a unipolar output configuration and a bipolar detection configuration. Where insulation and/or fracture issues arise for a proximal conductor (bipolar detection), an evoked response may not be sensed and, in turn, result in delivery of a high voltage back-up pulse and a ramping up of the primary output voltage (e.g., energy via voltage, pulse width, etc.). A capture threshold history may exhibit some information that relates to such a problem. In particular, a history may help to identify intermittent problems (e.g., sporadic increases in reported capture threshold where the actual unipolar capture threshold is relatively stable).

In clinical follow-up, a care provider may perform a threshold test to determine if the algorithm for capture is working properly and for further assessment. In systems that use the AutoCapture™ algorithm, a follow-up clinical test includes automatically and temporarily setting PV delay and AV delay intervals to about 25 ms and about 50 ms, respectively. Shortening of the AV and PV delays acts to minimize risk of fusion. Fusion (of any type) may compromise measurement and detection of an ER signal, especially ER signal amplitude. If results from the follow-up test indicate that enabling of the algorithm would not be safe due to too low an evoked response or too high a polarization signal, then the algorithm may be disabled and a particular, constant output programmed to achieve capture with a suitable safety margin. If the ER and polarization signals are appropriate to allow a capture algorithm to be enabled, an ER sensitivity will be recommended by the programmer and may then be programmed as it relates to detection of an ER signal.

The follow-up tests typically work top down. If loss of capture occurs, a first output adjustment step typically sets a high output and then decreases output by about 0.25 volts until loss of capture occurs (also referred to as a “down threshold”). At this point, output is increased in steps of a lesser amount (e.g., about 0.125 volts) until capture occurs. Once capture occurs, a working or functional margin of about 0.25 volts is added to the capture threshold output value. Hence, the final output value used is the capture threshold plus a working margin. Systems that use a fixed output use a safety margin ratio instead of an absolute added amount. The safety margin is a multiple of the measured capture threshold, commonly 2:1 or 100% to allow for fluctuations in the capture threshold between detailed evaluations at the time of office visits.

With respect to a down threshold approach, in instances where loss of capture occurs, a first output adjustment step typically increases output until capture is restored. Steps used in the AutoCapture™ algorithm are typically finer than those used in a routine follow-up capture threshold test. At times, a down threshold algorithm may result in a threshold that is as much as 1 volt lower from the result of an up threshold algorithm. This has been termed a Wedensky effect. In general, an actual output setting (e.g., including safety margin) may be adjusted to account for whether a patient is pacemaker dependent. In a patient who is not dependent on the pacing system, a narrower safety margin may be selected than would be the case for a patient whom the physician considers to be pacemaker dependent.

As already mentioned, lead instability may affect capture threshold, similarly, capture threshold history may help to identify lead instability. Lead instability includes issues germane to failure as well as issues germane to movement of a lead (e.g., to cause movement of an electrode of the lead, etc.). A stable capture threshold history may indicate normal lead function. However, marked fluctuations in capture threshold over time may indicate a lead stability problem, such as movement and variations with the degree of contact between the electrode and myocardial tissue. If the problem is associated with movement, repositioning or re-anchoring may be required. If such fluctuations occur in the early post-implant period, the problem may relate to positional instability as opposed to a marked inflammatory reaction at the electrode-tissue interface (e.g., “lead maturation”).

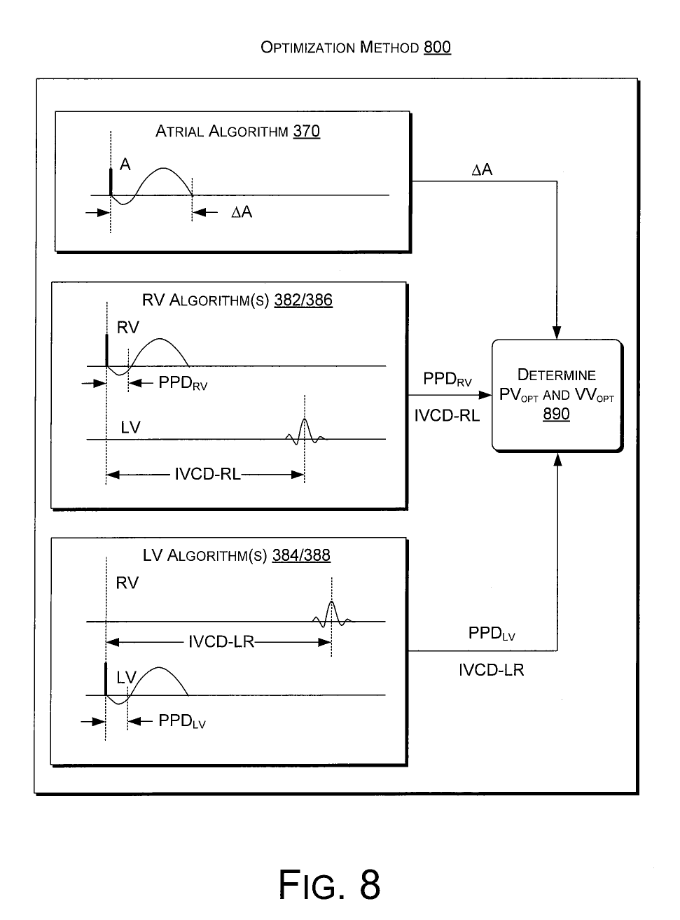

FIG. 8 shows an optimization method 800 for optimizing one or more timing parameters for bi-ventricular pacing therapy. While the method 800 refers to bi-ventricular pacing therapy, such a method may be suitably adapted for multi-site pacing therapy in a single ventricle (e.g., for a LV1 site and a LV2 site instead of a RV site and a LV site).

The method 800 includes the atrial algorithm 370 of FIG. 3 for acquiring atrial information, including an atrial wave width (ΔA). The method 800 also includes the right ventricular algorithms 382 and 386 of FIG. 3 for acquiring right ventricular information, for example, paced propagation delay at a right ventricular pacing site (PPD_{RV}) and/or an interventricular conduction delay (IVCD-RL). The method 800 also includes the left ventricular algorithms 384 and 388 of FIG. 3 for acquiring left ventricular information, for example, paced propagation delay at a left ventricular pacing site (PPD_{LV}) and/or an interventricular conduction delay (IVCD-LR). As shown in FIG. 8, a determination block 890 (see, e.g., the block 490 of FIG. 4) receives the information acquired using the algorithms 370, 382, 386, 384 and 388 to determine an optimal PV (or AV) and an optimal VV. The determination block 890 may rely on one or more blocks of the method 500 of FIG. 5 and/or one or more of the equations of the method 1300 of FIG. 13.

As described herein, an exemplary method can measure an IVCD while performing a ventricular capture assessment. For example, such a method may program a short AV (e.g., 50 ms), deliver a stimulus to one ventricle and sense activity in the other ventricle where the time between delivery of the stimulus and sensed activity responsive to the stimulus is the measured IVCD. In this example, the delivered stimulus is of sufficient energy to cause an evoked response.

With respect to the atrial information (e.g., ΔA), while not shown in the method 500 of FIG. 5, it may be used to determine an optimal PV or AV as shown in FIG. 13. In particular, such atrial information may be used to optimize PV or AV when a patient's activity state changes. For example, an optimal AV value may be determined as explained with respect to the method 500 of FIG. 5 and then the value may be further optimized using atrial information, especially where a patient's activity state changes.

With respect to the paced propagation delay information (PPD), an exemplary algorithm may determine PPD for the right ventricle (for a right ventricular lead) and for the left ventricle (for a left ventricular lead) during measurement of IVCD-LR and IVCD-RL (e.g., parameters that may be used to determine VV). Alternatively, where circumstances confound measurement of IVCD-LR and/or IVCD-RL, PPD may be measured for a right ventricle and a left ventricle and the difference used as a surrogate for the parameter Δ_{IVCD}.

While paced propagation delay can be measured from the time of delivering a pacing pulse to the time of an evoked response at the pacing lead (PPD_{-I}), paced propagation delay may be measured alternatively from the time of the pulse to the peak of an evoked response (PPD_{-Peak}). In either instance, such techniques may shorten block and/or discharge periods, optionally to a minimum (e.g., about 3 ms in some commercial ICDs).

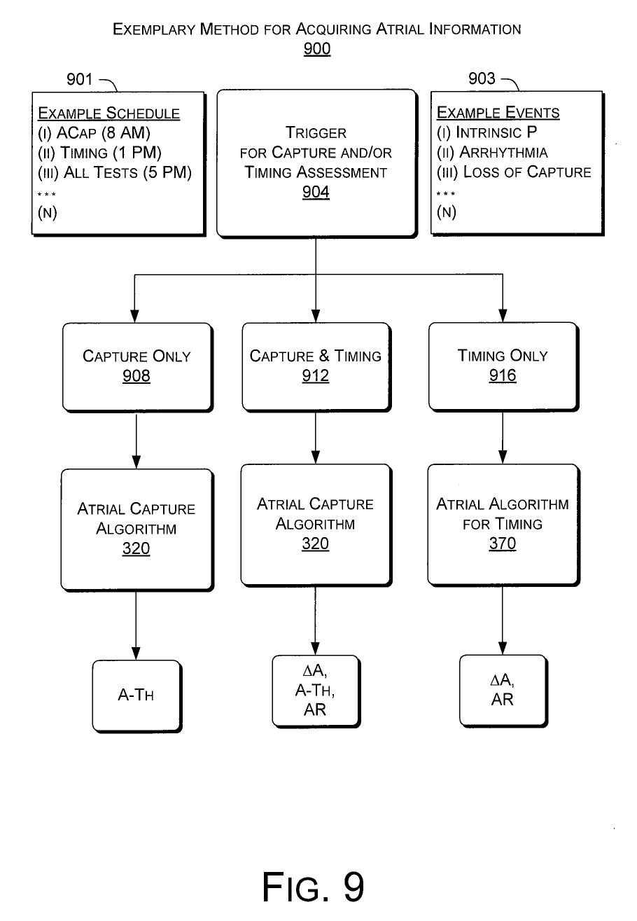

FIG. 9 shows an exemplary method 900 for acquiring atrial information for capture assessment, timing assessment or a combination of both capture assessment and timing assessment. The method 900 commences in a trigger block 904 for triggering capture assessment and/or timing assessment. The block 904 may trigger based on a schedule, an event or a combination of a schedule and an event. A schedule block 901 shows some examples of scheduled items while an event block 903 shows some examples of event items. Accordingly, the block 904 decides whether the method 900 should proceed to a capture only branch, a capture and timing branch or a timing only branch.

The capture only branch commences in a capture only block 908. The capture only block 908 calls for implementation of the atrial capture algorithm 320 to acquire an atrial threshold, for example, for use in setting an energy level for atrial pacing.

The capture and timing branch commences in a capture and timing block 912. The capture and timing block 912 calls for implementation of the atrial capture algorithm 320 to acquire an atrial threshold (e.g., for use in setting an energy level for atrial pacing), an atrial width ΔA (e.g., for use in optimizing a timing parameter for pacing the heart) and AR times for one or both ventricles (e.g., AR_{RV} and/or AR_{LV}) (e.g., depending on the requirements for determination of a timing parameter value or values).

The timing only branch commences in a timing only block 916. The timing only block 916 calls for implementation of the atrial algorithm for timing 370 to acquire an atrial width ΔA (e.g., for use in optimizing a timing parameter for pacing the heart) and AR times for one or both ventricles (e.g., AR_{RV} and/or AR_{LV}) (e.g., depending on the requirements for determination of a timing parameter value or values).

In an exemplary method, during atrial capture assessment tests, atrial paced signals in an ER sensing window are used to test whether the atrial paces captured the atrium, for example, by PDI, a Kendall tau algorithm, etc., with minimum blocking/discharge end. The atrial paced signals can also be used to determine ΔA, which can be used for optimizing one or more timing parameters.

As described herein, an exemplary method includes performing an atrial capture assessment, determining an atrial evoked response width (ΔA) using information acquired during the atrial capture assessment and optimizing an atrio-ventricular (PV or AV) delay based at least in part on the atrial evoked response width (ΔA). In such a method the information acquired during the atrial capture assessment may be a cardiac electrogram (e.g., an IEGM). As explained below, the optimizing can optimize the atrio-ventricular delay (AV or PV) with respect to a patient activity state (AS) (see, e.g., FIG. 13). As explained, the performing and the optimizing can occur at substantially the same time. Such a method may be embodied on one or more processor-readable media as processor-executable instructions.

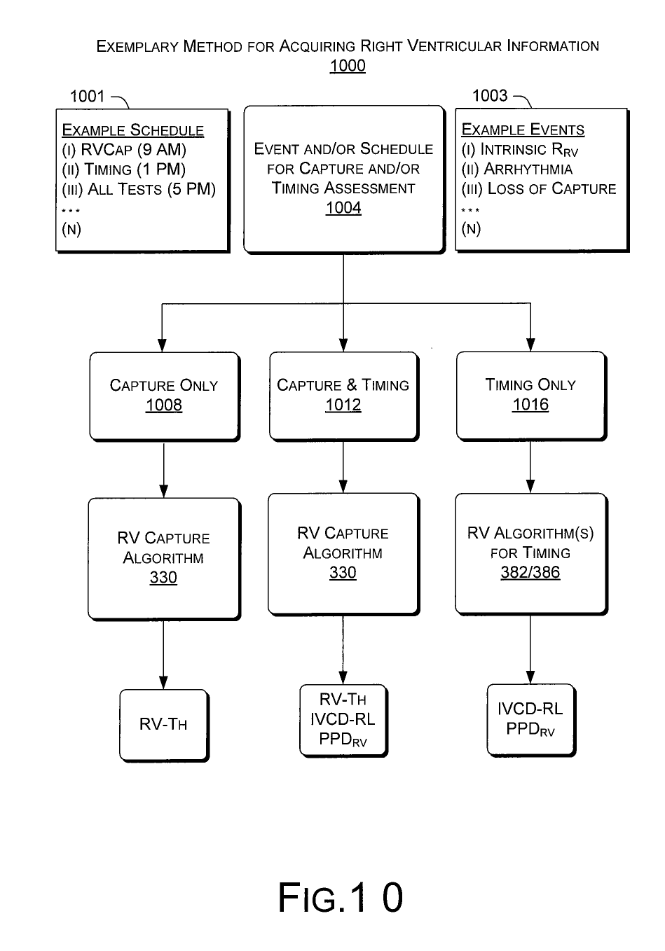

FIG. 10 shows an exemplary method 1000 for acquiring right ventricular information for capture assessment, timing assessment or a combination of both capture assessment and timing assessment. The method 1000 commences in a trigger block 1004 for triggering capture assessment and/or timing assessment. The block 1004 may trigger based on a schedule, an event or a combination of a schedule and an event. A schedule block 1001 shows some examples of scheduled items while an event block 1003 shows some examples of event items. Accordingly, the block 1004 decides whether the method 1000 should proceed to a capture only branch, a capture and timing branch or a timing only branch.

The capture only branch commences in a capture only block 1008. The capture only block 1008 calls for implementation of the right ventricular capture algorithm 330 to acquire a right ventricular threshold, for example, for use in setting an energy level for right ventricular pacing.

The capture and timing branch commences in a capture and timing block 1012. The capture and timing block 1012 calls for implementation of the right ventricular capture algorithm 330 to acquire a right ventricular threshold (e.g., for use in setting an energy level for right ventricular pacing), a right ventricular to left ventricular IVCD (IVCD-RL) and/or a right ventricular paced propagation delay (PPD_{RV}) (e.g., the latter two for use in optimizing a timing parameter for pacing the heart).

The timing only branch commences in a timing only block 1016. The timing only block 1016 calls for implementation of one or more right ventricular algorithm for timing 382 and/or 386 to acquire a right ventricular to left ventricular IVCD (IVCD-RL) and/or a right ventricular paced propagation delay (PPD_{RV}) for use in optimizing a timing parameter for pacing the heart.

With respect to the capture scenarios of FIG. 10, during RV capture tests, PV or AV delays are typically set short (e.g., about 50 ms) to avoid fusion beats and PDI or Dₘₐₓ or other techniques are used to detect capture with minimum block/discharge end. According to the method 1000, with this configuration, the signals acquired can be used to calculate paced propagation delay at the RV delivery site (PPD_{RV}) and/or the IVCD from paced RV to sensed LV (IVCD-RL).

As described herein, an exemplary method includes performing a right ventricular capture assessment, determining a right ventricular paced propagation delay (PPD_{RV}) using information acquired during the right ventricular capture assessment and optimizing an interventricular delay (VV) based at least in part on the right ventricular paced propagation delay (PPD_{RV}). Such a method may further include determining a left ventricular paced propagation delay (PPD_{LV}) and optimizing the interventricular delay (VV) based at least in part on the right ventricular paced propagation delay (PPD_{RV}) and the left ventricular paced propagation delay (PPD_{LV}). Such a method may be embodied on one or more processor-readable media as processor-executable instructions.

As described herein, an exemplary method includes performing a right ventricular capture assessment, determining an interventricular conduction delay from the right ventricle to the left ventricle (IVCD-RL) using information acquired during the right ventricular capture assessment and optimizing an interventricular delay (VV) based at least in part on the interventricular conduction delay from the right ventricle to the left ventricle (IVCD-RL). Such a method may further include determining an interventricular conduction delay from the left ventricle to the right ventricle (IVCD-LR) and optimizing the interventricular delay (VV) based at least in part on the interventricular conduction delay from the right ventricle to the left ventricle (IVCD-RL) and the interventricular conduction delay from the left ventricle to the right ventricle (IVCD-LR). Such a method may be embodied on one or more processor-readable media as processor-executable instructions.

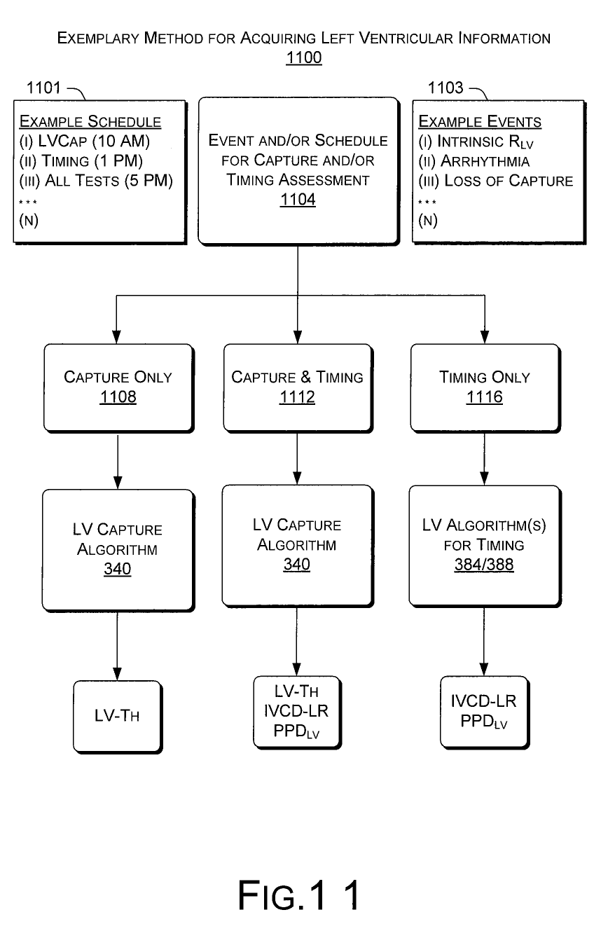

FIG. 11 shows an exemplary method 1100 for acquiring left ventricular information for capture assessment, timing assessment or a combination of both capture assessment and timing assessment. The method 1100 commences in a trigger block 1104 for triggering capture assessment and/or timing assessment. The block 1104 may trigger based on a schedule, an event or a combination of a schedule and an event. A schedule block 1101 shows some examples of scheduled items while an event block 1103 shows some examples of event items. Accordingly, the block 1104 decides whether the method 1100 should proceed to a capture only branch, a capture and timing branch or a timing only branch.

The capture only branch commences in a capture only block 1108. The capture only block 1108 calls for implementation of the left ventricular capture algorithm 340 to acquire a left ventricular threshold, for example, for use in setting an energy level for left ventricular pacing.

The capture and timing branch commences in a capture and timing block 1112. The capture and timing block 1112 calls for implementation of the left ventricular capture algorithm 340 to acquire a left ventricular threshold (e.g., for use in setting an energy level for left ventricular pacing), a left ventricular to right ventricular IVCD (IVCD-LR) and/or a left ventricular paced propagation delay (PPD_{LV}) (e.g., the latter two for use in optimizing a timing parameter for pacing the heart).

The timing only branch commences in a timing only block 1116. The timing only block 1116 calls for implementation of one or more left ventricular algorithm for timing 384 and/or 388 to acquire a left ventricular to right ventricular IVCD (IVCD-LR) and/or a left ventricular paced propagation delay (PPD_{LV}) for use in optimizing a timing parameter for pacing the heart.

With respect to the capture scenarios of FIG. 11, during LV capture tests, PV or AV delays are typically set short (e.g., about 50 ms) to avoid fusion beats and PDI or Dₘₐₓ or other techniques are used to detect capture with minimum block/discharge end. According to the method 1100, with this configuration, the signals acquired can be used to calculate paced propagation delay at the LV delivery site (PPD_{LV}) and/or the IVCD from paced LV to sensed RV (IVCD-LR).

As described herein, an exemplary method includes performing a left ventricular capture assessment, determining a left ventricular paced propagation delay (PPD_{LV}) using information acquired during the left ventricular capture assessment and optimizing an interventricular delay (VV) based at least in part on the left ventricular paced propagation delay (PPD_{LV}). Such a method may be embodied on one or more processor-readable media as processor-executable instructions.

As described herein, an exemplary method includes performing a left ventricular capture assessment, determining an interventricular conduction delay from the left ventricle to the right ventricle (IVCD-LR) using information acquired during the left ventricular capture assessment and optimizing an interventricular delay (VV) based at least in part on the interventricular conduction delay from the left ventricle to the right ventricle (IVCD-LR). Such a method may be embodied on one or more processor-readable media as processor-executable instructions.

As mentioned, various techniques can be used for multisite activation or pacing of a ventricle. For example, the left ventricle may be activated at two or more sites where an optimization algorithm determines the timing of energy delivered to the sites consider, for example, AV1_{Opt} and AV2_{Opt} and V1V2_{Opt} for a scheme that paces a ventricle using two sites.

In another scenario, a lead may include a series of electrodes where some of the electrodes may be better suited for delivery of energy than others for purposes of optimizing contraction of a ventricle or ventricles.

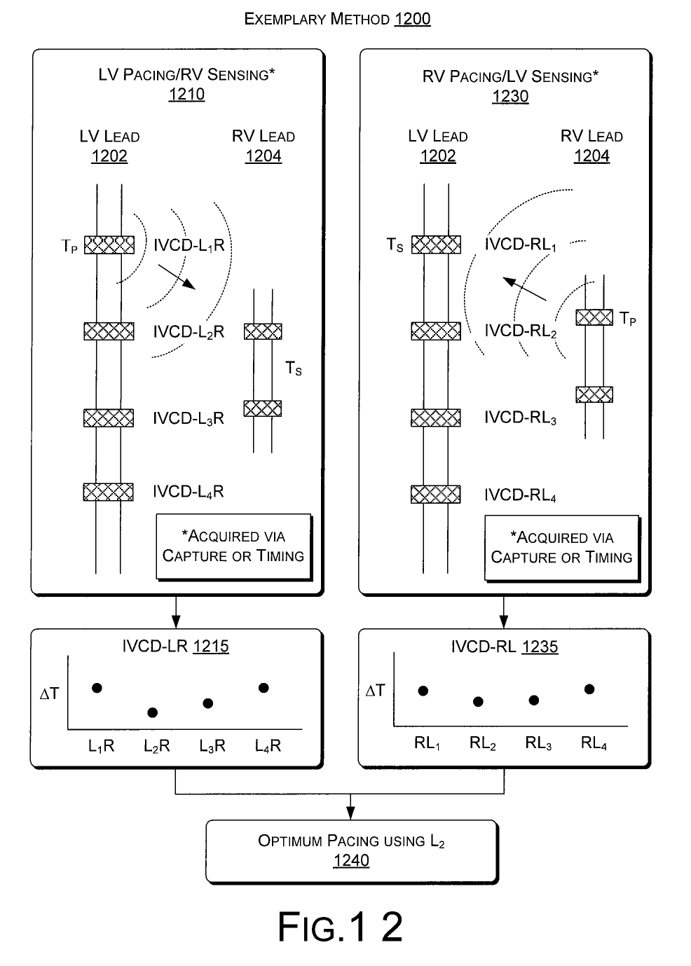

FIG. 12 shows an exemplary method 1200 where multiple activation sites exist in the left ventricle and where a single site is selected for delivery of energy to a ventricle. Specifically, in the example of FIG. 12, a quadrupolar left ventricular lead 1202 and a bipolar right ventricular lead 1204 are used. A LV pacing and RV sensing block 1210 illustrates a wave front propagating from LV lead electrode L₁ (e.g., unipolar energy delivery) to the RV sensing electrodes (e.g., bipolar sensing); thus, corresponding to measurement of IVCD-L₁R. An RV pacing and LV sensing block 1230 illustrates a wave front propagating from the RV electrodes (e.g., bipolar energy delivery) to the LV lead electrode L₁ (e.g., unipolar sensing); thus, corresponding to measurement of IVCD-RL₁.

In the example of FIG. 12, IVCD measurements are made. As described herein, such measurements may be made as part of a capture assessment. Consider a capture assessment for each of the electrodes of the quadrupolar left ventricular lead 1202. Each of these individual capture assessments may be used to acquire information for an IVCD-LR measure (IVCD-L₁R, IVCD-L₂R, etc.) and/or information for a paced propagation delay (e.g., PPD_{LV1}, PPD_{LV2}, etc.).

A plot 1215 shows IVCD-LR as a time delay (ΔT) versus energy delivery/sensing configuration while a plot 1235 shows IVCD-RL as a time delay (ΔT) versus energy delivery/sensing configuration. The data of plots 1215 and 1235 may be used in a determination block 1240 to determine optimum electrode configuration for LV pacing. In the example of FIG. 12, single site pacing using LV lead electrode L₂ corresponds to the shortest interventricular conduction delay.

In an alternative example, more than one electrode of the LV lead 1202 may be used to define a first site and a second site. Further, stimulation energy may be delivered at different times to the first site and the second site to active the myocardium in an optimal manner.

FIG. 13 shows various exemplary methods 1300. While equations are presented, implementation of techniques described herein may be implemented using any of a variety of forms of control logic. For example, look-up tables may be used together with logic that stores and/or pulls data from the look-up table. Control logic to achieve the overall goals achieved by the various equations 1300 may be achieved by control logic that does not explicitly rely on the equations, as presented.

A state block 1310 defines various activity states. The activity states include a base state, for example, a rest state denoted by a subscript “0”. In other examples, the subscript “rest” is used. The activity states include at least two states, for example, a base state and another activity state. In FIG. 13, the states range from the base state to activity state “N”, which may be an integer without any numeric limitation (e.g., N may equal 5, 10, 100, 1000, etc.). The number of activity states may depend on patient condition and patient activity. For example, a patient that is bedridden may have few activity states when compared to a young patient (e.g., 40 years old) fitted with a pacemaker that leads an active life with a regular exercise regimen.

A PV or AV states block 1320 presents equations for the parameters β and δ as well as for a base state PV and AV and PV and AV for a state other than a base activity state, referred to as ASₓ, where x=1, 2, . . . N. In addition, sets of equations are presented that include a paced propagation delay term PPD. A paced propagation delay may be a pacing latency, which is generally defined as the time between delivery of a cardiac stimulus and time of an evoked response caused by the stimulus. More specifically, an implantable device may use the time of delivery of a stimulus and the time at which a sensed, evoked response signal deviates from a baseline, which is referred to herein as PPD_{-I} (e.g., to initiation of evoked response). Such a signal is usually sensed using the lead that delivered the stimulus, however, electrode configuration may differ (e.g., unipolar delivery and bipolar sensing, bipolar delivery and unipolar sensing, etc.). In some instances, a PPD_{-I} may exceed 100 ms due to ischemia, scarring, infarct, etc. Thus, PV or AV timing may be adjusted accordingly to call for earlier delivery of a stimulus to a ventricle or ventricles.

An exemplary algorithm may determine PPD for the right ventricle (for a right ventricular lead) and PPD for the left ventricle (for a left ventricular lead) during measurement of IVCD-LR and IVCD-RL (e.g., parameters that may be used to determine VV). While paced propagation delay can be measured from the time of delivering a pacing pulse to the time of an evoked response at the pacing lead (PPD_{-I}), paced propagation delay may be measured alternatively, for example, from the time of the pulse to the peak of an evoked response (PPD_{-Peak}) (noting that other possibilities exist). For purposes of measurement, techniques may shorten block and/or discharge periods, optionally to a minimum (e.g., about 3 ms in some commercial ICDs). An algorithm may also provide for detection of capture, for example, using an integral (e.g., PDI) and/or a derivative (e.g., Dₘₐₓ). In general, paced propagation delays for LV and RV leads correspond to situations where capture occurs. In yet another alternative, during P wave and PR measurement, a time delay from a marker of a sensed R event to the peak of a QRS complex may be measured and used as a correction term akin to paced propagation delay.

A VV states block 1330 presents equations for the parameters α, Δ and Δ_{IVCD} and VV for a base activity state (AS₀) and another activity state (ASₓ). In the equations of FIG. 13, there is a lack of absolute value operators for the parameter Δ, as such, the value of Δ can be used to determine whether the right ventricle or left ventricle is paced for single ventricle pacing or is the master for bi-ventricular pacing. If the Δ is less than 0 ms, then the right ventricle is paced or the master whereas if Δ is greater than 0 ms, then the left ventricle is paced or the master. For bi-ventricular pacing, the PV or AV state equation is used for the master ventricle and then the VV equation is used to determine timing of the slave ventricle. Hence, the control logic uses Δ to determine whether the PV or AV state equation will correspond to the left ventricle or the right ventricle.

The block 1330 also includes equations for a paced propagation delay differential, referred to as ΔPPD. This term may be calculated, for example, as the difference between PPD_{RV} and PPD_{LV}, and be a surrogate for Δ_{IVCD}. A criterion or criteria may be used to decide if a paced propagation delay correction term should be used in determining PV, AV or VV.

While various examples mention use of a “rest” state, a rest state may be a base state. Alternatively, a base state may be a state other than a rest state. For example, a base state may correspond to a low activity state where a patient performs certain low energy movements (e.g., slow walking, swaying, etc.) that may be encountered regularly throughout a patient's day. Thus, a base state may be selected as a commonly encountered state in a patient's waking day, which may act to minimize adjustments to PV, AV or VV. Further, upon entering a sleep state, a device may turn off adjustments to PV, AV or VV and assume sleep state values for PV, AV or W. Such decisions may be made according to a timer, a schedule, an activity sensor, etc.

An exemplary computing device may include control logic to assess cardiac condition based at least in part on information acquired from an implantable device where the information includes, for example, one or more CRT parameters and/or one or more rate adaptive pacing parameters or combinations thereof (e.g., α, Δ, IVCD-RL, IVCD-LR, Δ_{IVCD}, AV, PV, VV, response time, recovery time, A-Th, RV-Th, LV-Th, PPD, ΔPPD, etc.). The computing device may be the implantable device, or in other words, an implantable device may be capable of assessing patient condition and more particularly cardiac condition.

Various exemplary methods may be implementable wholly or to varying extent using one or more computer-readable media (or processor-readable media) that include processor-executable instructions for performing one or more actions. For example, the device 100 of FIG. 2 shows various modules associated with a processor 220. Hence, a module may be developed using an algorithm described herein. Such a module may be downloadable to an implantable device using a device programmer or may be incorporated into a device during manufacture by any of a variety of techniques. At times such instructions are referred to as control logic.

As described herein, the exemplary one or more coordination algorithms 305 of FIG. 3 can set a schedule or schedules for execution of various algorithms. Such scheduling may take advantage of synergies that exist between capture and timing algorithms, especially with respect to types of information required to make capture or timing decisions. For example, QuickOpt™ tests can be scheduled as a subset of bi-ventricular AutoCapture™ tests since AutoCapture™ tests are expected more frequently than QuickOpt™ tests. Consider a schedule where if QuickOpt™ tests are scheduled daily but AutoCapture™ tests are scheduled three times per day, the calculations for QuickOpt™ tests can be enabled to execute in accordance with one of the three per day scheduled AutoCapture™ tests.

FIG. 14 shows an exemplary system 1400 that includes the exemplary implantable device 100 of FIGS. 1 and 2, with processor 220 including one or more modules 1410, for example, that may be loaded via memory 260. A series of leads 104, 106 and 108 provide for delivery of stimulation energy and/or sensing of cardiac activity, etc., associated with the heart 102. Stylized bullets indicate approximate positions or functionality associated with each of the leads 104, 106 and 108. Other arrangements are possible as well as use of other types of sensors, electrodes, etc.

Memory 260 is shown as including the capture algorithms 310 of FIG. 3, the timing algorithms 360 of FIG. 3 and the coordination algorithms 305 of FIG. 3. Memory 260 also includes appropriate modules (e.g., processor-executable instructions) for performing various actions of the algorithms 310, 360 and 305, noting that part of a method may be performed using a device other than the implantable device 100. For example, for acquisition of ECG information, an ECG unit 1435 may be used, which optionally communicates with the device 100 or one or more other devices (e.g., the device 1430, 1440, etc.).

The system 1400 includes a device programmer 1430 having a wand unit 1431 for communicating with the implantable device 100. The programmer 1430 may further include communication circuitry for communication with another computing device 1440, which may be a server. The computing device 1440 may be configured to access one or more data stores 1450, for example, such as a database of information germane to a patient, an implantable device, therapies, etc.

The programmer 1430 and/or the computing device 1440 may include various information such as data and modules (e.g., processor-executable instructions) for performing various actions of associated with the algorithms 310, 360 and 305, noting that a particular implementation of a method may use more than one device.

The programmer 1430 optionally includes features of the commercially available 3510 programmer and/or the MERLIN™ programmer marketed by St. Jude Medical, Sylmar, Calif. The MERLIN™ programmer includes a processor, ECC (error-correction code) memory, a touch screen, an internal printer, I/O interfaces such as a USB that allows a device to connect to the internal printer and attachment of external peripherals such as flash drives, Ethernet, modem and WiFi network interfaces connected through a PCMCIA/CardBus interface, and interfaces to ECG and RF (radio frequency) telemetry equipment.

The wand unit 1431 optionally includes features of commercially available wands. As shown, the wand unit 1431 attaches to a programmer 1430, which enables clinicians to conduct implantation testing and performance threshold testing, as well as programming and interrogation of pacemakers, implantable cardioverter defibrillators (ICDs), emerging indications devices, etc.

During implant, a system such as a pacing system analyzer (PSA) may be used to acquire information, for example, via one or more leads. A commercially available device marketed as WANDA™ (St. Jude Medical, Sylmar, Calif.) may be used in conjunction with a programmer such as the MERLIN™ programmer or other computing device (e.g., a device that includes a processor to operate according to firmware, software, etc.). Various exemplary techniques described herein may be implemented during implantation and/or after implantation of a device for delivery of electrical stimulation (e.g., leads and/or pulse generator) and the types of equipment for acquiring and/or analyzing information may be selected accordingly.

The wand unit 1431 and the programmer 1430 allow for display of atrial and ventricular electrograms simultaneously during a testing procedure. Relevant test measurements, along with customizable implant data, can be displayed, stored, and/or printed in a comprehensive summary report for the patient's medical records and physician review and/or for other purposes.

In the example of FIG. 14, the data store 1450 may include information such as measures, values, scores, etc. Such information may be used by a model, in making a comparison, in making a decision, in adjusting a therapy, etc. Such information may be updated periodically, for example, as the device 100 (or other device(s)) acquires new information about a patient. The system 1400 is an example as other equipment, instructions, etc., may be used or substituted for features shown in FIG. 14.

As described herein, an exemplary implantable device includes a processor, memory and control logic to acquire information during a capture assessment and to optimize one or more cardiac resynchronization therapy (CRT) timing parameters using the acquired information. In such a device, the timing parameters can include at least one timing parameter selected from a group of atrio-ventricular timing parameters (PV or AV) and interventricular timing parameters (VV). Such a device may perform atrial capture assessment, right ventricular capture assessment and/or left ventricular capture assessment. In such a device, the control logic may include (e.g., in part) processor-executable instructions stored in the memory.

### CONCLUSION

Although exemplary methods, devices, systems, etc., have been described in language specific to structural features and/or methodological acts, it is to be understood that the subject matter defined in the appended claims is not necessarily limited to the specific features or acts described. Rather, the specific features and acts are disclosed as exemplary forms of implementing the claimed methods, devices, systems, etc.

What is claimed is:

1. A method implemented at least in part by an implantable device, the comprising:
performing an atrial capture assessment;
determining an atrial evoked response width (ΔA) using information acquired during the atrial capture assessment;
determining one or more atrio-ventricular intervals (AR) using information acquired during the atrial capture assessment; and
optimizing an atrio-ventricular delay (PV or AV) based at least in part on the atrial evoked response width (ΔA) and the one or more atrio-ventricular intervals (AR).

2. The method of claim 1 wherein the information acquired during the atrial capture assessment comprises a cardiac electrogram.

3. The method of claim 1 wherein the optimizing optimizes the atrio-ventricular delay (AV or PV) with respect to a patient activity state (AS).

4. The method of claim 1 wherein the performing and the optimizing occur at substantially the same time.

5. A method implemented at least in part by an implantable device, the method comprising:
performing a right ventricular capture assessment;
determining a right ventricular paced propagation delay (PPD_{RV}) using information acquired during the right ventricular capture assessment; and
optimizing an atrio-ventricular delay (AV) and an interventricular delay (VV) based at least in part on the right ventricular paced propagation delay (PPD_{RV}).

6. The method of claim 5 wherein the information acquired during the right ventricular capture assessment comprises a cardiac electrogram.

7. The method of claim 5 further comprising determining a left ventricular paced propagation delay (PPD_{LV}) and optimizing the atrio-ventricular delay (AV) and the interventricular delay (VV) based at least in part on the right ventricular paced propagation delay (PPD_{RV}) and the left ventricular paced propagation delay (PPD_{LV}).

8. A method implemented at least in part by an implantable device, the method comprising:
performing a left ventricular capture assessment;
determining a left ventricular paced propagation delay (PPD_{LV}) using information acquired during the left ventricular capture assessment; and
optimizing an atrio-ventricular delay (AV) and an interventricular delay (VV) based at least in part on the left ventricular paced propagation delay (PPD_{LV}).

9. The method of claim 8 wherein the information acquired during the left ventricular capture assessment comprises a cardiac electrogram.
